SEQUENCE LISTING (21 sequences)

SEQ ID NO: 1 - protein, 448 aa, synthetic construct
  source 1..448: note = H1L1 Heavy Chain #1
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
  source 1..214: note = H1L1 Light Chain
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
  source 1..119: note = H1L1 VH
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
  source 1..107: note = H1L1 VL
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
  source 1..8: note = H1L1/H3L3 - CDR H1
GFSFSSYG

SEQ ID NO: 6 - protein, 8 aa, synthetic construct
  source 1..8: note = H1L1/H3L3 - CDR H2
ISPSGSYF

SEQ ID NO: 7 - protein, 12 aa, synthetic construct
  source 1..12: note = H1L1/H3L3 - CDR H3
ARLPGFNPPFDH

SEQ ID NO: 8 - protein, 6 aa, synthetic construct
  source 1..6: note = H1L1/H3L3 - CDR L1
QNVGGN

SEQ ID NO: 9 - skipped

SEQ ID NO: 10 - protein, 9 aa, synthetic construct
  source 1..9: note = H1L1/H3L3 - CDR L3
LQYKNNPWT

SEQ ID NO: 11 - protein, 448 aa, synthetic construct
  source 1..448: note = H3L3 Heavy Chain
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
  source 1..214: note = H3L3 Light Chain
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
  source 1..119: note = H3L3 VH
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
  source 1..107: note = H3L3 VL
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

SEQ ID NO: 15 - protein, 112 aa, synthetic construct
  source 1..112: note = CD3zeta signaling domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 15 aa, synthetic construct
  source 1..15: note = (G4S) linker 1
GGGGSGGGGSGGGGS

SEQ ID NO: 17 - protein, 18 aa, synthetic construct
  source 1..18: note = linker 2
GSTSGSGKPGSGEGSTKG

SEQ ID NO: 18 - protein, 45 aa, synthetic construct
  source 1..45: note = CD8alpha hinge
TTTPAPRPPTPAPTIASQPLSLRPEACRPAAGGAVHTRGLDFACD

SEQ ID NO: 19 - protein, 24 aa, synthetic construct
  source 1..24: note = TM domain
IYIWAPLAGTCGVLLLSLVITLYC

SEQ ID NO: 20 - protein, 42 aa, synthetic construct
  source 1..42: note = costimulatory domain
KRGRKKLLYIFKQPFMRPVQTTQEEDGCSCRFPEEEEGGCEL

SEQ ID NO: 21 - protein, 448 aa, synthetic construct
  source 1..448: note = H1L1 - Heavy Chain #2
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0179170 A1
(43) Pub. Date: Jun. 5, 2025

(54) USE OF ANTI-CLAUDIN-1 ANTIBODIES TO INCREASE T CELL AVAILABILITY

(71) Applicant: Alentis Therapeutics AG, Allschwil (CH)

(72) Inventors: Alberto TOSO, Basel (CH); Roberto IACONE, Basel (CH); Geoffrey TEIXEIRA, Basel (CH); Markus MEYER, Neuenburg (DE)

(21) Appl. No.: 18/842,610

(22) PCT Filed: Mar. 8, 2023
(86) PCT No.: PCT/IB2023/052214

§ 371 Date: Aug. 29, 2024

Related U.S. Application Data
(60) Provisional application No. 63/317,885, filed on Mar. 8, 2022.

(51) Int. Cl. C07K 16/28; A61K 35/17; A61K 40/11; A61K 40/31; A61K 40/42; A61K 45/06; C12N 5/0783

(52) U.S. Cl. CPC C07K 16/28; A61K 35/17; A61K 40/11; A61K 40/31; A61K 40/4202; A61K 45/06; C12N 5/0636; A61K 2239/13; C07K 2317/24; C07K 2317/565; C12N 2510/00

(57) ABSTRACT

The present disclosure relates to a method of promoting T cell mediated anti-tumor activity in a subject having a fibrotic tumor, comprising administering an anti-Claudin-1 antibody to the subject.

### CROSS REFERENCE TO RELATED APPLICATIONS

This application claims the priority benefit of U.S. Provisional App. No. 63/317,885, filed Mar. 8, 2022, which is hereby incorporated by reference in its entirety.

### REFERENCE TO A SEQUENCE LISTING

The content of the electronically submitted sequence listing (Name: 4872_013PC01_Seglisting_ST26; Size: 24,527 bytes; and Date of Creation: Mar. 6, 2023) is herein incorporated by reference in its entirety.

### FIELD OF THE INVENTION

According to various aspects of this disclosure, the present disclosure relates to methods of promoting T cell anti-tumor activity.

### BACKGROUND OF THE INVENTION

Cancer therapy using immune checkpoint inhibitors has greatly advanced a physician's ability to treat a subject. However, many cancers have shown immune checkpoint inhibitor resistance. One phenotype associated with immune checkpoint inhibitor resistance is the lack of tumor T cell infiltration, also known as a “cold tumor” or “T cell exclusion” (see Shuyue W. et. al, Front. Immun., 12:690112 (2021); Christian et al., Front. Oncol., 11:712788 (October 2021)). Therefore, a need exists to develop therapies to treat cold tumors or T cell exclusion in a way that allows therapies to overcome immune checkpoint inhibitor resistance.

### BRIEF SUMMARY OF THE INVENTION

The present disclosure provides a method of promoting T cell mediated anti-tumor activity in a subject having a fibrotic tumor, comprising administering an anti-Claudin-1 antibody to the subject.

In some aspects, the method further comprises administering an immune checkpoint inhibitor.

In some aspects, provided herein is a method of treating cancer in a subject having a solid tumor comprising administering to the subject a therapeutically effective amount of an anti-Claudin-1 antibody and an immune checkpoint inhibitor. In some aspects, the anti-Claudin-1 antibody promotes T cell mediated anti-tumor activity in a tumor in the subject.

In some aspects, provided herein is a method of increasing therapeutic efficacy of an immune checkpoint inhibitor in a subject having a fibrotic tumor, comprising a) administering an anti-Claudin-1 antibody to the subject, wherein the anti-Claudin-1 antibody promotes T cell mediated anti-tumor activity in the fibrotic tumor; and b) administering the immune checkpoint inhibitor to the subject.

In some aspects, the anti-Claudin-1 antibody is administered prior to the administration of the immune checkpoint inhibitor.

In some aspects, the anti-Claudin-1 antibody and the immune checkpoint inhibitor are administered simultaneously or sequentially.

In some aspects, the anti-Claudin-1 antibody and the immune checkpoint inhibitor are administered in the same composition.

In some aspects, the anti-Claudin-1 antibody and the immune checkpoint inhibitor are administered in different compositions.

In some aspects, the anti-Claudin-1 antibody and/or the immune checkpoint inhibitor are administered intratumorally, intravenously, intraperitoneally, intramuscularly, intrathecally or subcutaneously.

In some aspects, the immune checkpoint inhibitor is an antagonist of PD-1, PD-L1, CTLA-4, LAG-3, TIM-3, TIGIT, VISTA, B7-H3, BTLA, and/or Siglec-15.

In some aspects, the immune checkpoint inhibitor is a small molecule inhibitor.

In some aspects, the immune checkpoint inhibitor is an antibody.

In some aspects, the immune checkpoint inhibitor is a PD-1 antagonist selected from the group consisting of nivolumab, pembrolizumab, cemiplimab, and dostarlimab.

In some aspects, the immune checkpoint inhibitor is a PD-L1 antagonist selected from the group consisting of atezolizumab, durvalumab, and avelumab.

In some aspects, the immune checkpoint inhibitor is a CTLA-4 antagonist is selected from the group consisting of ipilimumab and tremelimumab.

In some aspects, the immune checkpoint inhibitor is a TIGIT antagonist selected from the group consisting of tiragolumab, ociperlimab, domvanalimab, etigilimab, and vibostolimab.

In some aspects, the cancer comprises a fibrotic tumor.

In some aspects, the fibrotic tumor is characterized by a high expression of Claudin-1 relative to a reference sample.

In some aspects, the reference sample is a tissue sample from a normal tissue, wherein the normal tissue is adjacent to the tumor.

In some aspects, the tumor is selected from the group consisting of a head and neck, a lung, a breast, a melanoma, a colorectal, a pancreatic, an esophageal, a cholangiocarcinoma, and a hepatocellular tumor.

In some aspects, the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

In some aspects, the anti-Claudin-1 antibody comprises a heavy chain sequence comprising the amino acid sequence set forth as SEQ ID NO: 1.

In some aspects, the anti-Claudin-1 antibody comprises a light chain sequence comprising the amino acid sequence set forth as SEQ ID NO: 2.

In some aspects, provided herein is a method of promoting T cell mediated anti-tumor activity in a subject having a fibrotic tumor, comprising administering an anti-Claudin-1 chimeric antigen receptor (CAR) T Cell to the subject. In some aspects, comprising administering an immune checkpoint inhibitor.

In some aspects, provided herein is a method of treating cancer in a subject having a solid tumor comprising administering to the subject a therapeutically effective amount of an anti-Claudin-1 CAR T Cell and an immune checkpoint inhibitor. In some aspects, the anti-Claudin-1 CAR T cell promotes T cell mediated anti-tumor activity in a tumor in the subject.

In some aspects, provided herein is a method of increasing therapeutic efficacy of an immune checkpoint inhibitor in a subject having a fibrotic tumor, comprising administering an anti-Claudin-1 CAR T Cell to the subject and administering an immune checkpoint inhibitor to the subject. In some aspects, the anti-Claudin-1 CAR T cell promotes T cell mediated anti-tumor activity in the fibrotic tumor.

### DESCRIPTION OF FIGURES

FIGS. 1A-1B (FIGS. 1A-1B) show Claudin-1 (CLDN1) expression in two different fibrotic tumor types. Immunohistochemistry (IHC) staining was performed to measure Claudin-1 expression (left panels) and fibrosis markers (right panels).

FIGS. 2A-2C (FIGS. 2A-2C) show T cell exclusion in Head and Neck cancer. FIG. 2A shows IHC staining for CLDN1 in a number of tumor samples, with the corresponding staining for CD3 that exemplifies a fibrotic trap. FIG. 2B shows the percentage of tumor samples that exhibited CLDN1 expression. FIG. 2C shows the breakdown of immune phenotypes for tumors with varying levels of CLDN1 expression. The immune phenotypes are hot (immune cells in the stroma and between cancer cells), excluded (immune cells mainly in the stroma) and cold (almost no immune cells visible).

FIGS. 3A-3D (FIGS. 3A-3D) show T cell exclusion in esophageal cancer. FIG. 3A shows the immune phenotypes for tumors with various levels of CLDN1 expression. The immune phenotypes are hot (immune cells in the stroma and between cancer cells), excluded (immune cells within the tumor, but only in the stroma) and cold (almost no immune cells visible). IHC staining was performed on an esophageal tumor sample for CLDN1 (FIG. 3B), T cells (CD3 staining) (FIG. 3C), and fibrotic tissue (Sirius Red staining) (FIG. 3D).

FIGS. 4A-4B (FIGS. 4A-4B) show immune escape (FIG. 4A) and T-cell exclusion (FIG. 4B) driven by over expression of mouse CLDN1 harboring human Extracellular loop in liver mouse tumor cells Hepa 1-6 in vivo.

FIG. 5 (FIG. 5) shows the volume of Hepa 1-6 tumors after inoculation. Samples positive for mouse CLDN1 harboring human extracellular loop (CLDN1 hECL) showed larger tumor volume and exhibited immune evasion as compared to samples with no CLDN1 expression.

FIG. 6 (FIG. 6) shows an exemplary diagram for to break checkpoint inhibitor resistance in cancer using anti-CLDN1 antibodies that have a direct anti-fibrotic effect.

FIG. 7A (FIG. 7A) shows overall survival probability over time in tumors (such as Melanoma) with high levels or low levels of CLDN1 after administration of the checkpoint inhibitor aPD1.

FIG. 7B (FIG. 7B) shows time to treatment discontinuation of time in patients that are CLDN1 positive or CLDN1 negative.

FIG. 8A (FIG. 8A) shows a mechanistic model for CLDN-1 targeting therapies in Tumor microenvironment.

FIG. 8B (FIG. 8B) shows a mechanistic model showing the effect of anti-CLDN1 antibody administration to tumor with T-cell exclusion.

FIG. 9 (FIG. 9) shows CLDN1 expression in Head and Neck Squamous Cell Carcinoma (HNSCC), Colorectal Cancer (CRC), Esophageal Cancer, Squamous Non-Small Cell Lung Cancer (Sq. NSCLC), Intrahepatic Cholangiocarcinoma (iCCA), Hepatocellular Carcinoma (HCC), and Urothelial Cancer.

FIG. 10A (FIG. 10A) shows the tumor volume in Hepa1-6 CLDN1+ challenged mice after administration with anti-CLDN1 antibody alone, PD1 antagonist (aPD1) alone, and a combination of anti-CLDN1 antibody and PD1 antagonist. Mice were rechallenged at 40 days.

FIGS. 10B-10C (FIGS. 10B-10C) show immunocytochemistry measuring T-cell dendritic cell infiltration in control (FIG. 10B) and after treatment with anti-CLDN1 antibody and PD1 antagonist (FIG. 10C).

### DETAILED DESCRIPTION OF THE INVENTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “treating” or “treatment” as used herein refers to the administration of a composition to a subject for therapeutic purposes.

The term “human Claudin-1 (or CLDN1)” refers to a protein having the sequence shown in NCBI Accession Number NP_066924.1, or any naturally occurring variants commonly found in HCV permissive human populations. The claudins are a family of approximately 18 proteins that play important structural and functional roles in tight junctions. They are transmembrane proteins that interact with other transmembrane proteins, such as junctional adhesion molecule (JAM) and occludin, as well as the scaffolding proteins ZO-1, ZO-2, and ZO-3. Claudin-1 expression is widespread among epithelial cells, but in mesenchymal tissues, expression appears limited to perineurial cells. Claudin-1 expression has been reported to occur in 29% to 92% of tumors.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×10⁷ M¹, and binds to the predetermined antigen with an affinity that is at least two-fold greater than the affinity for binding to a non-specific antigen (e.g., BSA, casein).

The term “monoclonal antibody” or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal antibody” or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, a “monoclonal antibody” or antigen-binding fragment thereof refers to such antibodies and antigen-binding fragments thereof made in any number of manners including but not limited to by hybridoma, phage selection, recombinant expression, and transgenic animals.

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains, in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

It is understood that wherever aspects are described herein with the language “comprising,” otherwise analogous aspects described in terms of “consisting of” and/or “consisting essentially of” are also provided.

As used herein, the term “administering” refers to the physical introduction of a composition comprising a therapeutic agent (e.g., combination of an anti-Claudin-1 antibody and/or an immune checkpoint inhibitor) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-Claudin-1 antibody and the amount of an immune checkpoint inhibitor, in combination, clinically proven to affect a significant decrease in cancer or slowing of progression of cancer, such as an advanced solid tumor.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “tumor” as used herein refers to any mass of tissue that results from excessive cell growth or proliferation, either benign (non-cancerous) or malignant (cancerous), including pre-cancerous lesions.

The term “cold tumor” as used herein refers to tumors that exhibit low levels of immune infiltrate, respond poorly to immunotherapies, and/or exhibit tumor heterogeneity. Such cold tumors can have features including, but not limited to, a substantial reduction in numbers and/or activities or absence of intratumoral CD8+T effector cells and/or substantial increase in numbers and/or activities of intratumoral immune suppressor cells. Cold tumors are also referred to as tumors with “T cell exclusion”. Immunotherapy by immune checkpoint inhibitors (ICIs), has greatly improved the clinical efficacy of malignant tumor therapy, but ICI-mediated antitumor responses depend on the infiltration of T cells capable of recognizing and killing tumor cells. Therefore ICIs may not be effective in “cold tumors”, which are characterized by the lack of T-cell infiltration.

The term “high expression of Claudin-1” refers to the proportion of cells in a test tissue sample that is scored as expressing Claudin-1. In some aspects, Claudin-1 expression is assayed by immunohistochemistry (IHC), where the high expression of Claudin-1 of a sample means that at least about 50%, at least about 60%, at least about 70%, at least about 80%, at least about 90%, or 100% of the total number of cells in the test sample express Claudin-1.

A “patient” as used herein includes any patient who is afflicted with a cancer (e.g., a fibrotic cancer). The terms “subject” and “patient” are used interchangeably herein.

### II. Anti-Claudin-1 Antibodies

The present invention concerns the use of anti-Claudin-1 antibodies for promoting T cell mediated anti-tumor activity in a subject having a fibrotic tumor, treating cancer in a subject having a solid tumor, and increasing therapeutic efficacy of an immune checkpoint inhibitor in a subject having a fibrotic tumor.

Antibodies directed against human Claudin-1 have been previously described to treat hepatitis c virus infection, hepatocellular carcinoma, and certain fibrotic diseases, such as lung fibrosis (see WO 2010/034812, WO 2016/146809, and WO 2021/094469). Anti-Claudin-1 antibodies that can be used in the practice of the present invention include any antibody raised against Claudin-1. Examples are disclosed in WO 2010/034812 and WO 2017/162678.

Other examples of suitable anti-Claudin-1 antibodies include those disclosed in European Patent No. EP 1 167 389, in U.S. Pat. No. 6,627,439, in international patent application published under No. WO 2014/132307, in international patent applications published under No. WO 2015/014659 and No. WO 2015/014357, and in Yamashita et al., J. Pharmacol. Exp. Ther., 2015, 353(1): 112-118.

Anti-Claudin-1 antibodies suitable for use in the present invention may be polyclonal antibodies or monoclonal antibodies.

Anti-Claudin-1 antibodies suitable for use according to the present invention may also be “humanized”: sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

In some aspects, a humanized anti-Claudin-1 antibody for use according to the present invention is one previously described in WO 2017/162678. Exemplary sequences for the antibody or antigen binding fragment provided herein are described in Table 1.

TABLE 1
Antibody or antigen binding fragments

| Description | Sequence | SEQ ID NO: |
|---|---|---|
| H1L1- | EVQLVESGGGLVKPGGSLRLSCAASGFSFSSYGMNWVRQA | 1 |
| Heavy | PGKGLEWVSSISPSGSYFYYADSVKGRFTISRDNAKNSLYLQ | |
| Chain #1 | MNSLRAEDTAVYYCARLPGFNPPFDHWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPELLGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |
| H1L1- | DIQMTQSPATLSVSPGERATLSCKASQNVGGNVDWYQWKP | 2 |
| Light Chain | GQAPRLLIYGASNRYTGIPARFRGSGSGTEFTLTISSLQSEDF | |
| | AVYYCLQYKNNPWTFGQGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| H1L1-VH | EVQLVESGGGLVKPGGSLRLSCAASGFSFSSYGMN | 3 |
| | WVRQAPGKGLEWVSSISPSGSYFYYADSVKGRFTI | |
| | SRDNAKNSLYLQMNSLRAEDTAVYYCARLPGFNP | |
| | PFDHWGQGTLVTVSS | |
| H1L1-VL | DIQMTQSPATLSVSPGERATLSCKASQNVGGNVD | 4 |
| | WYQWKPGQAPRLLIYGASNRYTGIPARFRGSGSG | |
| | TEFTLTISSLQSEDFAVYYCLQYKNNPWTFGQGT | |
| | KVEIK | |
| H1L1/H3L3- | GFSFSSYG | 5 |
| CDR H1 | | |
| H1L1/H3L3- | ISPSGSYF | 6 |
| CDR H2 | | |
| H1L1/H3L3- | ARLPGFNPPFDH | 7 |
| CDR H3 | | |
| H1L1/H3L3- | QNVGGN | 8 |
| CDR L1 | | |
| H1L1/H3L3- | GA | — |
| CDR L2 | | |
| H1L1/H3L3- | LQYKNNPWT | 10 |
| CDR L3 | | |
| H3L3- | QVQLVESGGGVVQPGRSLRLSCLGSGFSFSSYGMNWVRQA | 11 |
| Heavy | PGKGLEWVASISPSGSYFYYADSVKGRFTISRDNSKNTLY | |
| Chain | LQMTSLRAEDTAIYYCARLPGFNPPFDHWGQGTLVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKRVEPKSCDKTHTCPPCPAPELLGGP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALPAPIEKTISKAKGQPREPQVYTLPPSREEMTK | |
| | NQVSLTCLVKGFYPSDIAVEWESNGQPENNYKTTPPVL | |
| | DSDGSFFLYSKLTVDKSRWQQGNVFSCSVMHEALHNHYTQ | |
| | KSLSLSPG | |
| H3L3- | DIQMTQSPSSLSASVGDRVTITCKASQNVGGNVDWYQWKP | 12 |
| Light | GKAPKLLIYGASNRYTGVPDRFRGSGSGTDFTLTISSLQP | |
| Chain | EDVATYYCLQYKNNPWTFGGGTKVEIKRTVAAPSVFIFPPS | |
| | DEQLKSGTASVVCLLNNFYPREAKVQWKVDNALQSGNSQ | |
| | ESVTEQDSKDSTYSLSSTLTLSKADYEKHKVYACEVTHQGL | |
| | SSPVTKSFNRGEC | |
| H3L3-VH | QVQLVESGGGVVQPGRSLRLSCLGSGFSFSSYGM | 13 |
| | NWVRQAPGKGLEWVASISPSGSYFYYADSVKGR | |
| | FTISRDNSKNTLYLQMTSLRAEDTAIYYCARLPG | |
| | FNPPFDHWGQGTLVTVSS | |
| H3L3-VL | DIQMTQSPSSLSASVGDRVTITCKASQNVGGNV | 14 |
| | DWYQWKPGKAPKLLIYGASNRYTGVPDRFRGS | |
| | GSGTDFTLTISSLQPEDVATYYCLQYKNNPWTF | |
| | GGGTKVEIK | |
| HIL1 - | EVQLVESGGGLVKPGGSLRLSCAASGFSFSSYGMNWVRQA | 21 |
| Heavy | PGKGLEWVSSISPSGSYFYYADSVKGRFTISRDNAKNSLYLQ | |
| Chain #2 | MNSLRAEDTAVYYCARLPGFNPPFDHWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPEFEGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPASIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

In some aspects, the complementarity determining regions (CDRs) disclosed herein are defined according to IMGT©. However, it is appreciated that other methods of defining the CDRs in the art can also be used.

In some aspects, the six complementarity determining regions (CDRs) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a heavy chain variable region (“VH”) comprising the amino acid sequence set forth as SEQ ID NO: 3 or 13.

In some aspects, the anti-Claudin-1 antibody comprises a light chain variable region (“VL”) comprising the amino acid sequence set forth as SEQ ID NO: 4 or 14.

In some aspects, the heavy chain variable region (“VH”) and the light chain variable region (“VL”) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain and light chain of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a heavy chain sequence comprising the amino acid sequence set forth as SEQ ID NO: 1.

In some aspects, the anti-Claudin-1 antibody comprises a light chain sequence comprising the amino acid sequence set forth as SEQ ID NO: 2.

In some aspects, the anti-Claudin-1 antibody comprises a heavy chain sequence comprising the amino acid sequence set forth as SEQ ID NO: 1, SEQ ID NO: 11, or SEQ ID NO: 21.

In some aspects, the anti-Claudin-1 antibody comprises a light chain sequence comprising the amino acid sequence set forth as SEQ ID NO: 2 or SEQ ID NO: 12.

The humanized anti-Claudin-1 antibody may be a full monoclonal antibody having an isotope selected from the group consisting of IgG1, IgG2, IgG3 and IgG4. Alternatively, the humanized anti-Claudin-1 antibody may be a fragment of a monoclonal antibody selected from the group consisting of Fv, Fab, F(ab′)₂, Fab′, dsFv, scFv, sc(Fv)₂ and diabodies.

Anti-Claudin-1 antibodies (or biologically active variants or fragments thereof) suitable for use according to the present invention may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, NY; and Wilchek and Bayer, Anal. Biochem., 1988, 171: 1-32). Preferably, molecular entities are attached at positions on the antibody molecule that do not interfere with the binding properties of the resulting conjugate, e.g., positions that do not participate in the specific binding of the antibody to its target.

The antibody molecule and molecular entity may be covalently, directly linked to each other. Or, alternatively, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present invention is conjugated to a detectable agent. Any of a wide variety of detectable agents can be used, including, without limitation, various ligands, radionuclides (e.g., ³H, ¹²⁵I, ¹³¹I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and aequorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present invention (or a biologically active fragment thereof) include, but are not limited to, linear or branched hydrophilic polymeric groups, fatty acid groups, or fatty ester groups.

Thus, in the practice of the present invention, anti-Claudin-1 antibodies can be used under the form of full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)2 fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent or a detectable agent. Preferably, anti-Claudin-1 antibody-related molecules according to the present invention retain the antibody's ability to bind its antigen, in particular the extracellular domain of Claudin-1.

### III. Chimeric Antigen Receptors

Chimeric antigen receptor (CAR) T-cell therapy, or CAR T-cell therapy, is a cancer treatment, based on the use of T cells genetically engineered to express a synthetic receptor that binds a tumor antigen. Engineered CAR T cells are expanded in vitro and infused into the patient's body to attack and destroy chemotherapy-resistant cancer.

The term “chimeric antigen receptor” (CAR) refers to molecules that combine a binding domain against a component present on the target cell, for example an antibody-based specificity for a desired antigen (e.g., a tumor antigen, such as CLDN-1) with a T cell receptor-activating intracellular domain to generate a chimeric protein that exhibits a specific anti-target cellular immune activity.

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD5, CD22, CD79a, CD79b and CD66d. In some aspects, the signal transducing domain of the CAR can comprise the CD3zeta signaling domain (SEQ ID NO: 15).

CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy chain variable fragments of a monoclonal antibody joined by a flexible linker. This molecule is joined to an intracellular signaling molecule, comprising one or more intracellular signaling domains that mediate T-cell activation. The signaling domains for first generation CARs are derived from the cytoplasmic region of the CD3zeta or the Fc receptor gamma chains (or the intracellular signaling domain of another immune-receptor-tyrosine-based-activation-motif [ITAM]-containing protein). First generation CARs have been shown to successfully redirect T-cell cytotoxicity. However, they failed to provide prolonged expansion and anti-tumor activity in vivo. Signaling domains from co-stimulatory molecules, as well as transmembrane and hinge domains have been added to form CARs of second, third, and fourth generations. Second generation chimeric receptors also incorporate a co-stimulatory endodomain (e.g., 4-1BB/CD3ζ). Third generation CARs containing multiple co-stimulatory signaling modules. Fourth-generation CARs were generated by adding IL-12 to the base of the second-generation constructs, and are known as T cell redirected for universal cytokine-mediated killing (TRUCKs). TRUCKs augment T-cell activation and activate and attract innate immune cells to eliminate antigen-negative cancer cells in the targeted lesion. Therapeutic trials in humans using CAR T-cell therapy have shown some success. For example, CAR redirected T cells specific for the B cell differentiation antigen CD19 have shown dramatic efficacy in the treatment of B cell malignancies, while TCR-redirected T cells have shown benefits in patients suffering from solid cancer. Stauss et al. describe strategies to modify therapeutic CARs and TCRs, for use in the treatment of cancer, for example, to enhance the antigen-specific effector function and limit toxicity of engineered T cells (Current Opinion in Pharmacology 2015, 24:113-118).

In some aspects of the present invention includes a chimeric antigen receptor (CAR) which is specific for Claudin-1, which is expressed on the surface of cancer cells. In some aspects of the present invention, a CAR as described herein comprises an extracellular target-specific binding domain, a transmembrane domain, an intracellular signaling domain (such as a signaling domain derived from CD3zeta or FcRgamma), and/or one or more co-stimulatory signaling domains derived from a co-stimulatory molecule, such as, but not limited to, 4-1BB. In some aspects, the CAR includes a hinge or spacer region between the extracellular binding domain and the transmembrane domain, such as a CD8alpha hinge. In some aspects, the chimeric antigen receptor (CAR) comprises an extracellular target-specific binding domain, which is an anti-Claudin single chain antibody (scFv), and may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86: 3833-3837. Thus, in some aspects, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In some aspects, the CARs of the present invention may comprise a linker between the various domains, added for appropriate spacing and conformation of the molecule. For example, in some aspects, there may be a linker between the binding domain VH or VL which may be between 1-10 amino acids long. In some aspects, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In some aspects, the linker may be 21, 22, 23, 24, 25, 26, 27, 28, 29 or 30 amino acids long. Ranges including the numbers described herein are also included herein, e.g., a linker 10-30 amino acids long.

In some aspects, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure. Specific linkers include (G4S)n linkers, wherein n=1-3. In some aspects, the linker comprises the amino acid sequence of SEQ ID NO: 16 or SEQ ID NO: 17.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In some aspects, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered. In some aspects, the hinge region comprises a CD8alpha hinge (SEQ ID NO: 18).

The “transmembrane” region or domain is the portion of the CAR that anchors the extracellular binding portion to the plasma membrane of the immune effector cell, and facilitates binding of the binding domain to the target antigen. The transmembrane domain may be a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In some aspects, the transmembrane domain is the transmembrane domain of CD137. In some aspects, the transmembrane domain comprises the amino acid sequence of SEQ ID NO: 19. In some aspects, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRγ chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the invention include those derived from TCRzeta, FcRgamma, FcRbeta, CD3gamma, CD3delta, CD3epsilon, CD5, CD22, CD79a, CD79b and CD66d. In one some aspects, the intracellular signaling domain of the anti-BCMA CARs described herein are derived from CD3zeta. In some aspects, the signaling domain comprises the amino acid sequence of SEQ ID NO: 15.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-1BB, other costimulatory domains are contemplated for use with the CARs described herein. The inclusion of one or more co-stimulatory signaling domains may enhance the efficacy and expansion of T cells expressing CAR receptors. The intracellular signaling and costimulatory signaling domains may be linked in any order in tandem to the carboxyl terminus of the transmembrane domain. In some aspects, the costimulatory domain comprises the amino acid sequence of SEQ ID NO: 20.

In some aspects, the anti-Claudin-1 CARs of the present disclosure comprise any of the elements of Table 2.

TABLE 2
CAR elements

| Description | Sequence | SEQ ID NO: |
|---|---|---|
| CD3zeta | RVKFSRSADAPAYKQGQNQLYNELNLGRREEYDVLDKRR | 15 |
| signaling | GRDPEMGGKPRRKNPQEGLYNELQKDKMAEAYSEIGMK | |
| domain | GERRRGKGHDGLYQGLSTATKDTYDALHMQALPPR | |
| (G4S) linker 1 | GGGGSGGGGSGGGGS | 16 |
| linker 2 | GSTSGSGKPGSGEGSTKG | 17 |
| CD8alpha | TTTPAPRPPTPAPTIASQPLSLRPEACRPAAGGAVHTRGL | 18 |
| hinge | DFACD | |
| TM domain | IYIWAPLAGTCGVLLLSLVITLYC | 19 |
| costimulatory | KRGRKKLLYIFKQPFMRPVQTTQEEDGCSCRFPEEEEGGC | 20 |
| domain | EL | |

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

In some aspects, the anti-Claudin-1 CAR of the invention comprises (a) an anti-Claudin-1 binding domain (e.g., an scFv having binding regions (e.g., CDRs or variable domains) from any one or more of the sequences identified in Table 1) (b) a hinge region derived from human CD8alpha, (c) a human CD8alpha transmembrane domain, and (d) a human T cell receptor CD3 zeta chain (CD3) intracellular signaling domain, and optionally one or more costimulatory signaling domains, e.g., 4-1BB. In some aspects, the different protein domains are arranged from amino to carboxyl terminus in the following order: an anti-Claudin-1 binding domain, a hinge region and a transmembrane domain. The intracellular signaling domain and optional co-stimulatory signaling domains are linked to the transmembrane carboxy terminus in any order in tandem to form a single chain chimeric polypeptide. In some aspects, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain and a CD3zeta intracellular signaling domain. In some aspects, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain, a 4-1BB co-stimulatory domain, and a CD3zeta co-stimulatory domain.

In some aspects, the polynucleotide encoding the CAR described herein is inserted into a vector. For expression of an anti-Claudin-1 CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

The CARs of the present invention are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In some aspects, the CAR of the present invention is introduced and expressed in immune effector cells so as to redirect their specificity to a target antigen of interest, e.g. Claudin-1.

The present invention provides methods for making the immune effector cells which express the CAR as described herein. In some aspects, the method comprises transfecting or transducing immune effector cells isolated from a subject, such as a subject having a Claudin-1 expressing tumor cell, such that the immune effector cells express one or more CAR as described herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject. In some aspects, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some aspects, T cell can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis may be washed to remove the plasma fraction and to place the cells in an appropriate buffer or media for subsequent processing. In some aspects, the cells are washed with PBS. In some aspects, the washed solution lacks calcium, and may lack magnesium or may lack many, if not all, divalent cations. As would be appreciated by those of ordinary skill in the art, a washing step may be accomplished by methods known to those in the art, such as by using a semi-automated flow through centrifuge. After washing, the cells may be resuspended in a variety of biocompatible buffers or other saline solution with or without buffer. In some aspects, the undesirable components of the apheresis sample may be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting may also be used to isolate cell populations of interest for use in the present invention.

PBMCs may be used directly for genetic modification with the CARs using methods as described herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells. In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+T helper cells can be sorted into naive, central memory, and effector cells by identifying cell populations that have cell surface antigens. CD4+ lymphocytes can be obtained by standard methods. In some aspects, naive CD4+T lymphocytes are CD45RO−, CD45RA+, CD62L+CD4+ T cell. In some aspects, central memory CD4+ cells are CD62L positive and CD45RO positive. In some aspects, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In some aspects, the immune effector cells, such as T cells, are genetically modified with the chimeric antigen receptors described herein (e.g., transduced with a viral vector comprising a nucleic acid encoding a CAR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells may be activated and stimulated to proliferate with feeder cells and appropriate antibodies and cytokines using methods such as those described in U.S. Pat. Nos. 6,040,177; 5,827,642; and WO2012129514.

The invention provides a population of modified immune effector cells for the treatment of a patient having a malignancy caused by a Claudin-1 expressing tumor, the modified immune effector cells comprising an anti-Claudin-1 CAR as disclosed herein.

### IV. Methods of Use

Methods of the present invention may be accomplished using an anti-Claudin-1 antibody, or a biologically active fragment thereof, or a pharmaceutical composition comprising such an antibody or fragment (see below). These methods generally comprise administration of an effective amount of an anti-Claudin-1 antibody, or biologically active fragment thereof, or of a pharmaceutical composition thereof, to a subject in need thereof (i.e., a subject having a fibrotic tumor). Administration may be performed using any of the administration methods known to one skilled in the art (see below).

Fibrotic tumors typically have a dense collagen network, which causes small interfibrillar spacing in the interstitium to retard the movement of particles larger than 10 nanometers (Netti P A, et al. (2000) Cancer Res 60:2497-2503; Pluen A, et al. (2001) Proc Natl AcadSci USA 98:4628-4633; Ramanujan S, et al. (2002) Biophys J 83:1650-1660; and Brown E, et al. (2003) Nat Med 9:796-800). In some aspects, provided herein is a method of promoting T cell mediated anti-tumor activity in a subject having a fibrotic tumor, comprising administering an anti-Claudin-1 antibody to the subject. In some aspects, the method of promoting T cell mediated anti-tumor activity in a subject having a fibrotic tumor further comprises administering an immune checkpoint inhibitor.

In some aspects, provided herein is a method of treating cancer in a subject having a solid tumor comprising administering to the subject a therapeutically effective amount of an anti-Claudin-1 antibody and an immune checkpoint inhibitor, wherein the anti-Claudin-1 antibody promotes T cell mediated anti-tumor activity in a tumor in the subject.

In some aspects, provided herein is a method of increasing therapeutic efficacy of an immune checkpoint inhibitor in a subject having a fibrotic tumor, comprising a) administering an anti-Claudin-1 antibody to the subject, wherein the anti-Claudin-1 antibody promotes T cell mediated anti-tumor activity in the fibrotic tumor; and b) administering the immune checkpoint inhibitor to the subject.

In some aspects, the anti-Claudin-1 antibody is administered prior to the administration of the immune checkpoint inhibitor.

In some aspects, the anti-Claudin-1 antibody and the immune checkpoint inhibitor are administered simultaneously or sequentially.

In some aspects, the anti-Claudin-1 antibody and the immune checkpoint inhibitor are administered in the same composition.

In some aspects, the anti-Claudin-1 antibody and the immune checkpoint inhibitor are administered in different compositions.

In some aspects, the anti-Claudin-1 antibody and/or the immune checkpoint inhibitor are administered intratumorally, intravenously, intraperitoneally, intramuscularly, intrathecally or subcutaneously.

In some aspects, the immune checkpoint inhibitor is an antagonist of PD-1, PD-L1, CTLA-4, LAG-3, TIM-3, TIGIT, VISTA, B7-H3, BTLA, and/or Siglec-15.

In some aspects, the immune checkpoint inhibitor is a small molecule inhibitor.

In some aspects, the immune checkpoint inhibitor is an antibody or antigen binding fragment thereof.

In some aspects, the immune checkpoint inhibitor is a PD-1 antagonist. In some aspects, the PD-1 antagonist is selected from the group consisting of nivolumab, pembrolizumab, cemiplimab, and dostarlimab.

In some aspects, the immune checkpoint inhibitor is a PD-L1 antagonist. In some aspects, the PD-L1 antagonist is selected from the group consisting of atezolizumab, durvalumab, and avelumab.

In some aspects, the immune checkpoint inhibitor is a CTLA-4 antagonist. In some aspects, the CTLA-4 antagonist is selected from the group consisting of ipilimumab and tremelimumab.

In some aspects, the immune checkpoint inhibitor is a LAG-3 antagonist (e.g. BI754111)

In some aspects, the immune checkpoint inhibitor is a TIM-3 antagonist (e.g., TSR-022 and LY3321367).

In some aspects, the immune checkpoint inhibitor is a VISTA (V-domain immunoglobulin (Ig)-containing suppressor of T-cell activation) antagonist (e.g., CA-170 (anti-PD-L1/L2 and anti-VISTA small molecule) and JNJ-61610588).

In some aspects, the immune checkpoint inhibitor is a B7-H3 antagonist.

In some aspects, the immune checkpoint inhibitor is a BTLA antagonist.

In some aspects, the immune checkpoint inhibitor is a Siglec-15 antagonist.

In some aspects, the immune checkpoint inhibitor is a TIGIT antagonist (e.g., BMS-986207, OMP-313M32, COM902 (CGEN-15137), and AB154). In some aspects, the TIGIT antagonist is selected from the group consisting of tiragolumab, ociperlimab, domvanalimab, etigilimab, and vibostolimab.

In some aspects, the cancer comprises a fibrotic tumor.

In some aspects, the fibrotic tumor is characterized by a high expression of Claudin-1. In some aspects, the methods disclosed herein further comprise detecting Claudin-1 expression levels in a fibrotic tumor sample from a subject. In some aspects, the methods disclosed herein further comprise comparing the Claudin-1 expression levels to the expression level of Claudin-1 in a reference sample, wherein if the expression levels of Claudin-1 in the fibrotic tumor sample are increased with respect to the expression levels of Claudin-1 in a reference sample, then the subject is administered the anti-Claudin-1 antibody and/or the immune checkpoint inhibitor as described herein.

In some aspects, the Claudin-1 expression levels in the fibrotic tumor sample and/or the reference sample are quantified via immunohistochemistry (IHC) test. In some aspects, the IHC test is calculated via the H-score method (see e.g., Parris, Toshima Z et al., BMC cancer vol. 14:324 (2014)). In some aspects, the H-score requires each cell to receive a score of 0 to +3 (0=negative staining; +1=weak staining or low expression of the target antigen; +2=moderate staining or medium expression of the target antigen; and +3=strong staining or high expression of the target antigen). In some aspects, the H-score ranges from 0 to 300, where the H-Score is calculated by adding i) percentage of cells in a sample with a score of +1, ii) two times the percentage of cells in a sample with a score of +2, and iii) three times the percentage of cells in a sample with a score of +3 (i.e., H-score=(1*% of cells with 1+)+(2*% of cells with 2+)+(3*% of cells with 3+)). In some aspects, a high expression of Claudin-1 as graded by the H-score method is between about 150 and about 300. In some aspects, a medium expression of Claudin-1 as graded by the H-score method is between about 50 and about 149. In some aspects, a low expression of Claudin-1 as graded by the H-score method is between about 1 and about 49.

In some aspects, expression of Claudin-1 is considered positive when the H-score is between about 1 and about 300. In some aspects, expression of Claudin-1 is considered positive when the H-score is between about 50 and about 300. In some aspects, expression of Claudin-1 is considered positive when the H-score is between about 150 and about 300.

In order to improve the treatment of fibrotic tumors, in some aspects, the present invention provides identifying a patient as having a high expression of Claudin-1 and providing an immunotherapy of an anti-Claudin-1 antibody and an immune checkpoint inhibitor.

In another aspect, the present invention is directed to identifying a patient as having a fibrotic tumor with high expression of Claudin-1 and treating said fibrotic tumor by administering an anti-Claudin-1 antibody or a combination of an anti-Claudin-1 antibody and an immune checkpoint inhibitor. In some aspects, the invention includes a method of identifying a patient as having a fibrotic tumor with high expression of Claudin-1 and administering to the patient an anti-Claudin-1 antibody.

In some aspects, the invention includes a method of selecting a fibrotic tumor in a human patient for immunotherapy, comprising: (a) determining the level of Claudin-1 expression in a tumor sample; and (b) selecting the tumor for immunotherapy if the tumor sample has high expression of Claudin-1.

In some aspects, the invention includes a method of identifying a fibrotic tumor in a human patient as eligible for immunotherapy, comprising: (a) determining the level of Claudin-1 expression in a tumor sample; and (b) identifying the tumor as eligible for immunotherapy if the tumor sample has high expression of Claudin-1.

In some aspects, the invention includes a method of identifying a fibrotic tumor in a human patient that is likely to be responsive to an immunotherapy, the method comprising: (a) determining the level of Claudin-1 expression in a tumor sample; and (b) identifying the tumor as likely to be responsive to treatment if the tumor has a high expression of Claudin-1.

In some aspects, the invention includes a method of classifying a fibrotic tumor in a human patient as likely to be responsive to an immunotherapy, the method comprising: (a) determining the level of Claudin-1 expression in a tumor sample; and (b) classifying the tumor as likely to be responsive to immunotherapy if the tumor has high expression of Claudin-1. In some aspects, the immunotherapy comprises contacting the tumor with a therapeutically effective amount of an anti-Claudin-1 antibody and an immune checkpoint inhibitor.

In some aspects, the invention includes a method of identifying a patient with a fibrotic tumor who is likely to respond to an immunotherapy, the method comprising: (a) determining the level of Claudin-1 expression in a tumor sample; and (b) identifying the patient who is likely to respond to treatment if the tumor has high expression of Claudin-1.

In some aspects, the invention includes a method of selecting a patient with a fibrotic tumor for immunotherapy, the method comprising: (a) determining the level of Claudin-1 expression in a tumor sample; and (b) selecting the patient for immunotherapy if the tumor has high expression of Claudin-1. In some aspects, the immunotherapy comprises contacting the tumor with a therapeutically effective amount of an anti-Claudin-1 antibody and an immune checkpoint inhibitor.

In some aspects, the identifying comprises determining Claudin-1 expression in a fibrotic tumor.

In some aspects, Claudin-1 expression is determined by receiving the results of an assay capable of determining Claudin-1 expression.

In order to assess the Claudin-1 expression, in some aspects, a test tissue sample is obtained from the patient who is in need of the therapy. In some aspects, a test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen or cryoconserved tissue sample. In some aspects, the test tissue sample is a fresh frozen (FF) tissue (e.g., tumor) sample. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In another aspect, the assessment of Claudin-1 expression can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express Claudin-1 on the surface of the cells based on an assessment that the proportion of cells in the test tissue sample that express Claudin-1 on the cell surface is higher than a predetermined threshold level.

In any of the methods comprising the measurement of Claudin-1 expression in a test tissue sample, however, it should be understood that the step comprising the provision of a test tissue sample obtained from a patient is an optional step. That is, in certain aspects the method includes this step, and in other aspects, this step is not included in the method. It should also be understood that in certain aspects the “measuring” or “assessing” step to identify, or determine the number or proportion of, cells in the test tissue sample that express Claudin-1 is performed by a transformative method of assaying for Claudin-1 expression, for example by performing a reverse transcriptase-polymerase chain reaction (RT-PCR) assay or an IHC assay. In certain other aspects, no transformative step is involved and Claudin-1 expression is assessed by, for example, reviewing a report of test results from a laboratory. In some aspects, Claudin-1 expression is assessed by reviewing the results of an immunohistochemistry assay from a laboratory. In certain aspects, the steps of the methods up to, and including, assessing Claudin-1 expression provides an intermediate result that may be provided to a physician or other healthcare provider for use in selecting a suitable candidate for the combination therapy of a Claudin-1 inhibitor and an immune checkpoint inhibitor. In certain aspects, the steps of the methods up to, and including, assessing Claudin-1 expression provides an intermediate result that may be provided to a physician or other healthcare provider for use in selecting a suitable candidate for an immune checkpoint inhibitor therapy. In certain aspects, the steps that provide the intermediate result is performed by a medical practitioner or someone acting under the direction of a medical practitioner. In other aspects, these steps are performed by an independent laboratory or by an independent person such as a laboratory technician.

In certain aspects of any of the present methods, the proportion of cells that express Claudin-1 is assessed by performing an assay to detect the presence of Claudin-1 RNA. In further aspects, the presence of Claudin-1 RNA is detected by RT-PCR, in situ hybridization or RNase protection. In some aspects, the presence of Claudin-1 RNA is detected by an RT-PCR based assay. In some aspects, scoring the RT-PCR based assay comprises assessing the level of Claudin-1 RNA expression in the test tissue sample relative to a predetermined level.

In other aspects, the proportion of cells that express Claudin-1 is assessed by performing an assay to detect the presence of Claudin-1 polypeptide. In further aspects, the presence of Claudin-1 polypeptide is detected by IHC, enzyme-linked immunosorbent assay (ELISA), in vivo imaging, or flow cytometry. In some aspects, Claudin-1 expression is assayed by IHC. In other aspects of all of these methods, cell surface expression of Claudin-1 is assayed using, e.g., IHC or in vivo imaging.

In some aspects, the immunohistochemistry assay is scored at a low magnification. In some aspects, low magnification is about 20×. In some aspects, the immunohistochemistry assay is scored at high magnification. In some aspects, high magnification is about 40×.

In some aspects, the immunohistochemistry assay is scored by an image analysis software. In some aspects, the immunohistochemistry assay is scored by pathologist visual immune score. In some aspects, the immunohistochemistry assay is scored manually.

In some aspects, the tumor is selected from the group consisting of a head and neck, a lung, a breast, a melanoma, a colorectal, a pancreatic, an esophageal, a cholangiocarcinoma, and a hepatocellular tumor.

In some aspects, CAR-expressing immune effector cells prepared as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some aspects, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

In some aspects, the pharmaceutical composition comprises an anti-Claudin-1 CAR-T cell and an immune check point inhibitor. In some aspects, the immune checkpoint inhibitor is selected from the group consisting of CTLA-4, PD-1, PD-L1, PD-L2, B7-H3, B7-H4, HVEM, TIM-3, GAL-9, LAG-3, VISTA, KIR, BTLA, TIGIT, IDO, and/or Siglec-15 inhibitors, as described herein.

In some aspects, the immune checkpoint inhibitor is an inhibitor of PD-1, as described herein. In some aspects, the immune checkpoint inhibitor is an inhibitor of PD-L1, as described herein. In some aspects, the immune checkpoint inhibitor is an inhibitor of PD-L2, as described herein. In some aspects, the immune checkpoint inhibitor is an inhibitor of CTLA-4, as described herein. In some aspects, the immune checkpoint inhibitor is an inhibitor of LAG-3, as described herein. In some aspects, the immune checkpoint inhibitor is an inhibitor of TIM-3, as described herein. In some aspects, the immune checkpoint inhibitor is an inhibitor of TIGIT, as described herein. In some aspects, the immune checkpoint inhibitor is an inhibitor of VISTA, as described herein. In some aspects, the immune checkpoint inhibitor is an inhibitor of B7-H3, as described herein. In some aspects, the immune checkpoint inhibitor is an inhibitor of B7-H4, as described herein. In some aspects, the immune checkpoint inhibitor is an inhibitor of HVEM, as described herein. In some aspects, the immune checkpoint inhibitor is an inhibitor of GAL-9, as described herein. In some aspects, the immune checkpoint inhibitor is an inhibitor of KIR, as described herein. In some aspects, the immune checkpoint inhibitor is an inhibitor of BTLA, as described herein. In some aspects, the immune checkpoint inhibitor is an inhibitor of IDO, as described herein. In some aspects, the immune checkpoint inhibitor is an inhibitor of Siglec-15 as described herein.

In some aspects, the CAR expressing immune effector cell populations of the present invention may be administered either alone, or as a pharmaceutical composition in combination with diluents and/or with other components such as an immune checkpoint inhibitor as described herein. Briefly, pharmaceutical compositions of the present invention may comprise a CAR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

In some aspects, the anti-tumor immune response induced in a subject by administering CAR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present invention, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

In some aspects, provided herein is a method of promoting T cell mediated anti-tumor activity in a subject having a fibrotic tumor, comprising administering an anti-Claudin-1 chimeric antigen receptor (CAR) T Cell to the subject. In some aspects, comprising administering an immune checkpoint inhibitor.

In some aspects, provided herein is a method of treating cancer in a subject having a solid tumor comprising administering to the subject a therapeutically effective amount of an anti-Claudin-1 CAR T Cell and an immune checkpoint inhibitor. In some aspects, the anti-Claudin-1 CAR T cell promotes T cell mediated anti-tumor activity in a tumor in the subject.

In some aspects, provided herein is a method of increasing therapeutic efficacy of an immune checkpoint inhibitor in a subject having a fibrotic tumor, comprising administering an anti-Claudin-1 CAR T Cell to the subject and administering an immune checkpoint inhibitor to the subject. In some aspects, the anti-Claudin-1 CAR T cell promotes T cell mediated anti-tumor activity in the fibrotic tumor.

### V. Immune Checkpoint Inhibitors

Immune checkpoint proteins interact with specific ligands which send a signal into T-cells that inhibits T-cell function. Cancer cells exploit this by driving high level expression of checkpoint proteins on their surface thereby suppressing the anti-cancer immune response.

An immune checkpoint inhibitor comprises any compound capable of inhibiting the function of an immune checkpoint protein. Inhibition includes reduction of function as well as full blockade. In some aspects, the immune checkpoint protein is a human checkpoint protein. Thus, in some aspects, the immune checkpoint inhibitor is preferably an inhibitor of a human immune checkpoint.

In some aspects, checkpoint proteins include, without limitation, CTLA-4, PD-1 (and its ligands PD-L1 and PD-L2), B7-H3, B7-H4, HVEM, TIM-3, GAL-9, LAG-3, VISTA, KIR, BTLA, TIGIT, IDO, and/or Siglec-15. The pathways involving LAG-3, BTLA, B7-H3, B7-H4, TIM-3 and KIR constitute immune checkpoint pathways similar to the CTLA-4 and PD-1 dependent pathways (see e.g., Pardoll, 2012, Nature Rev Cancer 12:252-264; Mellman et al., 2011, Nature 480:480-489). In some aspects, the immune checkpoint inhibitor is an inhibitor of CTLA-4, PD-1, PD-L1, PD-L2, B7-H3, B7-H4, HVEM, TIM-3, GAL-9, LAG-3, VISTA, KIR, BTLA, TIGIT, IDO, and/or Siglec-15. In some aspects, the immune checkpoint inhibitor is an inhibitor of PD-1, PD-L1, CTLA-4, LAG-3, TIM-3, TIGIT, VISTA, B7-H3, BTLA, and/or Siglec-15.

In some aspects, the immune checkpoint inhibitor is an inhibitor of PD-1. In some aspects, the immune checkpoint inhibitor is an inhibitor of PD-L1. In some aspects, the immune checkpoint inhibitor is an inhibitor of CTLA-4. In some aspects, the immune checkpoint inhibitor is an inhibitor of LAG-3. In some aspects, the immune checkpoint inhibitor is an inhibitor of TIM-3. In some aspects, the immune checkpoint inhibitor is an inhibitor of TIGIT. In some aspects, the immune checkpoint inhibitor is an inhibitor of VISTA. In some aspects, the immune checkpoint inhibitor is an inhibitor of B7-H3. In some aspects, the immune checkpoint inhibitor is an inhibitor of BTLA. In some aspects, the immune checkpoint inhibitor is an inhibitor of Siglec-15.

In some aspects, the immune checkpoint inhibitor is an antibody.

In some aspects, immune checkpoint inhibitors comprise antibodies or fragments thereof that specifically bind to an immune checkpoint protein selected from the group consisting of CTLA-4, PD-1, PD-L1, PD-L2, B7-H3, B7-H4, HVEM, TIM-3, GAL-9, LAG-3, VISTA, KIR, BTLA, TIGIT, IDO, and Siglec-15. In some aspects, the immune checkpoint inhibitor is a monoclonal antibody, a fully human antibody, a chimeric antibody, a humanized antibody or fragment thereof that capable of at least partly antagonizing CTLA-4, PD-1, PD-L1, PD-L2, B7-H3, B7-H4, HVEM, TIM-3, GAL-9, LAG-3, VISTA, KIR, BTLA, TIGIT, IDO, and/or Siglec-15.

In some aspects, the immune checkpoint inhibitor is an antibody or fragment thereof that specifically binds to PD-1. In some aspects, the immune checkpoint inhibitor is an antibody or fragment thereof that specifically binds to PD-L1. In some aspects, the immune checkpoint inhibitor is an antibody or fragment thereof that specifically binds to CTLA-4. In some aspects, the immune checkpoint inhibitor is an antibody or fragment thereof that specifically binds to LAG-3. In some aspects, the immune checkpoint inhibitor is an antibody or fragment thereof that specifically binds to TIM-3. In some aspects, the immune checkpoint inhibitor is an antibody or fragment thereof that specifically binds to TIGIT. In some aspects, the immune checkpoint inhibitor is an antibody or fragment thereof that specifically binds to VISTA. In some aspects, the immune checkpoint inhibitor is an antibody or fragment thereof that specifically binds to B7-H3. In some aspects, the immune checkpoint inhibitor is an antibody or fragment thereof that specifically binds to BTLA. In some aspects, the immune checkpoint inhibitor is an antibody or fragment thereof that specifically binds to Siglec-15.

In some aspects, the pharmaceutical composition comprises an anti-Claudin-1 antibody, or a biologically active fragment thereof, and a CTLA-4 inhibitor, preferably a monoclonal antibody that specifically binds to (and inhibits) CTLA-4. The complete human CTLA-4 nucleic acid sequence can be found under GenBank Accession No. NG_011502.1. Monoclonal antibodies that specifically bind to CTLA-4 include, without limitation, Ipilimumab (Yervoy®; BMS) and Tremelimumab (AstraZeneca/MedImmune), as well as antibodies disclosed in U.S. Patent Application Publication Nos. 2005/0201994, 2002/0039581, and 2002/0086014, the contents of each of which are incorporated herein by reference, and antibodies disclosed in U.S. Pat. Nos. 5,811,097; 5,855,887; 6,051,227; 6,984,720; 6,682,736; 6,207,156; 5,977,318; 6,682,736; 7,109,003; 7,132,281; and 8,491,895 the contents of each of which are incorporated herein by reference, or an antibody comprising the heavy and light chain variable regions of any of these antibodies. Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. No. 7,034,121 and International Publication Nos. WO 2012/122444, WO 2007/113648, WO 2016/196237, and WO 2000/037504. In some aspects, the immune checkpoint inhibitor is a CTLA-4 antagonist. In some aspects, the CTLA-4 antagonist is selected from the group consisting of ipilimumab and tremelimumab.

In some aspects, the pharmaceutical composition comprises an anti-Claudin-1 antibody, or a biologically active fragment thereof, and a PD-1 inhibitor, preferably a monoclonal antibody that specifically binds to (and inhibits) PD-1. The complete nucleotide and amino acid sequences of human PD-1 can be found under GenBank Accession No. NG_012110.1 and NP_005009.2. In some aspects, the anti-PD-1 antibody is nivolumab. Nivolumab (also known as “OPDIVO©”; BMS-936558; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another aspect, the anti-PD-1 antibody or fragment thereof cross-competes with nivolumab. In other aspects, the anti-PD-1 antibody or fragment thereof binds to the same epitope as nivolumab. In certain aspects, the anti-PD-1 antibody has the same CDRs as nivolumab.

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-human-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121168, the teachings of which are hereby incorporated by reference, can be used. Other known PD-1 antibodies include lambrolizumab (MK-3475) described in WO 2008/156712, and AMP-514 described in WO 2012/145493, the teachings of which are hereby incorporated by reference. Further known anti-PD-1 antibodies and other PD-1 inhibitors include those described in WO 2009/014708, WO 03/099196, WO 2009/114335 and WO 2011/161699, the teachings of which are hereby incorporated by reference. In some aspects, the anti-PD-1 antibody is REGN2810. In some aspects, the anti-PD-1 antibody is PDR001. Another known anti-PD-1 antibody is pidilizumab (CT-011). Antibodies or antigen binding fragments thereof that compete with any of these antibodies or inhibitors for binding to PD-1 also can be used.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA©, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540), which references are herein incorporated by reference.

In another aspect, the anti-PD-1 antibody or antigen binding fragment thereof cross-competes with pembrolizumab. In some aspects, the anti-PD-1 antibody or antigen binding fragment thereof binds to the same epitope as pembrolizumab. In certain aspects, the anti-PD-1 antibody or antigen binding fragment thereof has the same CDRs as pembrolizumab. In another aspect, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In some aspects, the PD-1 antagonist is selected from the group consisting of nivolumab, pembrolizumab, cemiplimab, and dostarlimab.

In some aspects, the pharmaceutical composition comprises an anti-Claudin-1 antibody, or a biologically active fragment thereof, and a PD-L1 inhibitor, preferably a monoclonal antibody that specifically binds to (and inhibits) PD-L1. Any recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 101110, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Other examples of an anti-PD-L1 antibody include atezolizumab (TECENTRIQ; RG7446), or durvalumab (IMFINZI; MEDI4736) or avelumab (Bavencio). Antibodies or antigen binding fragments thereof that compete with any of these art-recognized antibodies or inhibitors for binding to PD-L1 also can be used.

In certain aspects, the anti-PD-L1 antibody is BMS-936559 (formerly 12A4 or MDX-1105) (see, e.g., U.S. Pat. No. 7,943,743; WO 2013/173223). In other aspects, the anti-PD-L1 antibody is MPDL3280A (also known as RG7446 and atezolizumab) (see, e.g., Herbst et al. 2013 J Clin Oncol 31 (suppl):3000; U.S. Pat. No. 8,217,149), MEDI4736 (Khleif, 2013, In: Proceedings from the European Cancer Congress 2013; September 27-Oct. 1, 2013; Amsterdam, The Netherlands. Abstract 802), or MSB0010718C (also called Avelumab; see US 2014/0341917). In certain aspects, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31 (suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)).

In some aspects, the pharmaceutical composition comprises an anti-Claudin-1 antibody, or a biologically active fragment thereof, and a PD-L2 inhibitor such as MIH18 (described in Pfistershammer et al., Eur J Immunol. 36:1104-1113 (2006).

In some aspects, the pharmaceutical composition comprises an anti-Claudin-1 antibody, or a biologically active fragment thereof, and a LAG-3 inhibitor. In some aspects, the LAG-3 inhibitor is an anti-LAG-3 antibody.

Anti-human-LAG-3 antibodies (or VH/VL domains derived therefrom) suitable for use in the invention can be generated using methods well known in the art. Alternatively, art recognized anti-LAG-3 antibodies can be used. For example, the anti-human LAG-3 antibody described in US2011/0150892 A1, the teachings of which are hereby incorporated by reference, and referred to as monoclonal antibody 25F7 (also known as “25F7” and “LAG3.1) can be used. Other art recognized anti-LAG-3 antibodies that can be used include IMP731 (H5L7BW) described in US 2011/007023, MK-4280 (28G-10) described in WO2016028672, REGN3767 described in Journal for ImmunoTherapy of Cancer, (2016) Vol. 4, Supp. Supplement 1 Abstract Number: P195, BAP050 described in WO2017/019894, IMP-701 (LAG-525), IMP321 (eftilagimod alpha)), Sym022, TSR-033, MGD013, B1754111, FS118, AVA-017 and GSK2831781. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO/2017/086419, WO2018/034227, and WO2014/140180. The contents of each of these references are herein incorporated by reference.

In some aspects, the pharmaceutical composition comprises an anti-Claudin-1 antibody, or a biologically active fragment thereof, and a BLTA inhibitor such as the antibody 4C7 disclosed in U.S. Pat. No. 8,563,694, incorporated herein by reference.

In some aspects, the pharmaceutical composition comprises an anti-Claudin-1 antibody, or a biologically active fragment thereof, and a B7-H4 checkpoint inhibitor such as an antibody as disclosed in U.S. Patent Application Publication No. 2014/0294861, incorporated herein by reference or a soluble recombinant form of B7-H4 e.g. as disclosed in U.S. Patent Application Publication No. 2012/0177645, incorporated herein by reference.

In some aspects, the pharmaceutical composition comprises an anti-Claudin-1 antibody, or a biologically active fragment thereof, and a B7-H3 checkpoint inhibitor such as the antibody MGA271 disclosed as BRCA84D or a derivative as disclosed in U.S. Patent Application Publication No. 2012/0294796, incorporated herein by reference.

In some aspects, the pharmaceutical composition comprises an anti-Claudin-1 antibody, or a biologically active fragment thereof, and a TIM-3 checkpoint inhibitor such as an antibody as disclosed in U.S. Pat. No. 8,841,418, incorporated herein by reference or the anti-human TIM-3 blocking antibody F38-2E2 disclosed by Jones et al., J. Exp. Med., 205(12):2763-79 (2008).

In some aspects, the pharmaceutical composition comprises an anti-Claudin-1 antibody, or a biologically active fragment thereof, and a KIR checkpoint inhibitor such as the antibody lirilumab (described in Romagne et al., Blood, 114(13):2667-2677 (2009)).

In some aspects, the pharmaceutical composition comprises an anti-Claudin-1 antibody, or a biologically active fragment thereof, and a TIGIT inhibitor. TIGIT checkpoint inhibitors preferably inhibit interaction of TIGIT with poliovirus receptor (CD155) and include, without limitation, antibodies targeting human TIGIT, such as those disclosed in U.S. Pat. No. 9,499,596 (incorporated herein by reference) and U.S. Patent Application Publication Nos. 2016/0355589, 2016/0176963 (incorporated herein by reference) and poliovirus receptor variants such as those disclosed in U.S. Pat. No. 9,327,014 (incorporated herein by reference). In some aspects, the immune checkpoint inhibitor is a TIGIT antagonist. In some aspects, the TIGIT antagonist is selected from the group consisting of tiragolumab, ociperlimab, domvanalimab, etigilimab, and vibostolimab.

In some aspects, the pharmaceutical composition comprises an anti-Claudin-1 antibody, or a biologically active fragment thereof, and an IDO inhibitor (Indoleamine-pyrrole 2,3-dioxygenase. IDO is recognized as an immune checkpoint protein its expression in tumor cells contributes to immune tolerance by shutting down effector T-cells. IDO is thought to contribute to resistance of anti-CTLA-4 therapies. In some aspects, inhibitors of IDO for use according to the methods described herein include, without limitation, tryptophan mimetics such as D-1MT (D isoform of 1-methyl-DL-tryptophan (MT)), L-1MT (L isoform of MT), MTH-Trp (methylthiohydantoin-dl-tryptophan; transcriptional suppressor of IDO), and β-carbolines, indole mimetics such as napthoquinone-based agents, S-allyl-brassinin, S-benzyl-brassinin, 5-Bromo-brassinin, as well as phenylimidazole-based agents, 4-phenylimidazole, exiguamine A, epacadostat, rosmarinic acid, norharmane and NSC401366. In some aspects, IDO inhibitors include INCB 024360 (epacadostat; N′-(3-bromo-4-fluorophenyl)-N-hydroxy-4-[2-(sulfamoylamino)ethylamino]-1,2,5-oxadiazole-3-carboximidamide), indoximod ((2R)-2-amino-3-(1-methylindol-3-yl)propanoic acid), IDO peptide vaccine (Copenhagen University) and NLG919 (NewLink Genetics; 1-cyclohexyl-2-(5H-imidazo[5,1-a]isoindol-5-yl)ethanol). In some aspects, IDO inhibitors preferably inhibit metabolic pathways and include, without limitation, Norharmane, (see, Chiarugi A, et al., “Combined inhibition of indoleamine 2,3-dioxygenase and nitric oxide synthase modulates neurotoxin release by interferon-gamma-activated macrophages”, Journal of Leukocyte Biology. 68 (2): 260-6. (2000)), rosmarinic acid (see, Lee H J, et al., “Rosmarinic acid inhibits indoleamine 2,3-dioxygenase expression in murine dendritic cells”, Biochemical Pharmacology. 73 (9): 1412-21 (2007)), COX-2 inhibitors (see, Cesario A, et al., “The interplay between indoleamine 2,3-dioxygenase 1 (IDO1) and cyclooxygenase (COX)-2 in chronic inflammation and cancer”, Current Medicinal Chemistry. 18 (15): 2263-71 (2011)), 1-methyltryptophan (Hou D Y, et al., “Inhibition of indoleamine 2,3-dioxygenase in dendritic cells by stereoisomers of 1-methyl-tryptophan correlates with antitumor responses”. Cancer Research. 67 (2): 792-801 (2007) and Chauhan N, et al., (April 2009), “Reassessment of the reaction mechanism in the heme dioxygenases”. Journal of the American Chemical Society. 131 (12): 4186-7 (2009)), including for example, the specific racemer 1-methyl-D-tryptophan (known as indoximod), Epacadostat (INCB24360), navoximod (GDC-0919) (see, Jochems C, et al., “The IDO1 selective inhibitor epacadostat enhances dendritic cell immunogenicity and lytic ability of tumor antigen-specific T cells”, Oncotarget. 7 (25): 37762-37772. (2016)), and or BMS-986205. In some aspects, the IDO inhibitor is selected from the group consisting of Norharmane, rosmarinic acid, COX-2 inhibitors, 1-methyl-tryptophan, Indoximod, Epacadostat (INCB24360), navoximod (GDC-0919) and/or BMS-986205.

In some aspects, the pharmaceutical composition comprises an anti-Claudin-1 antibody, or a biologically active fragment thereof, and a TIGIT inhibitor. TIGIT checkpoint inhibitors preferably inhibit interaction of TIGIT with poliovirus receptor (CD155) and include, without limitation, antibodies targeting human TIGIT, such as those disclosed in U.S. Pat. No. 9,499,596 (incorporated herein by reference) and U.S. Patent Application Publication Nos. 2016/0355589, 2016/0176963 (incorporated herein by reference) and poliovirus receptor variants such as those disclosed in U.S. Pat. No. 9,327,014 (incorporated herein by reference).

In some aspects, the immune checkpoint inhibitor is an antagonist of IDO1 (indoleamine-2,3-dioxygenase 1) (e.g., indoximod (NLG8189, 1-methyl-D-TRP), epacadostat (INCB-024360), KHK2455, PF-06840003 (PCT Publication No. WO 2016/181348 A1), pyrrolididine-2,5-dione derivatives (PCT Publication No. WO 2015/173764 A1), navoximod (RG6078, GDC-0919, NLG919), and BMS-986205 (F001287)); KIR (killer-cell immunoglobulin-like receptor) (e.g., lirilumab (I-7F9, BMS-980615, or IPH2101) and IPH4102 (an anti-KIR3DL2 monoclonal antibody); TDO (tryptophan 2,3-dioxygenase) (e.g., 4-(indol-3-yl)-pyrazole derivatives (U.S. Pat. No. 9,126,984 B2 and U.S. Publication No. 2016/0263087 A1); 3-indol substituted derivatives (PCT Publication Nos. WO 2015140717 A1, WO 2017025868 A1, WO 2016147144 A1), 3-(indol-3-yl)-pyridine derivatives (U.S. Publication No. 20150225367 A1 and PCT Publication No. WO 2015121812 A1); dual IDO/TDO (e.g., small molecule dual IDO/TDO inhibitors as disclosed in PCT Publication Nos. WO 2015150097 A1, WO 2015082499 A2, WO 2016026772 A1, WO 2016071283 A1, WO 2016071293 A2, and WO 2017007700 A1); CD40 (e.g., Lineage BMS3h-56 (U.S. Pat. No. 9,475,879), lucatumumab (HCD122 and CHIR-12.12), CHIR-5.9, and dacetuzumab (huS2C6, PRO 64553, RG 3636, SGN 14, SGN-40)); adenosine A2a receptor (A2aR) (e.g., CPI-444, PBF-509, istradefylline (KW-6002), preladenant (SCH420814), tozadenant (SYN115), vipadenant (BIIB014), HTL-1071, ST1535, SCH412348, SCH442416, SCH58261, ZM241385, and AZD4635 (a small molecule A2aR inhibitor)); CEACAM1 (CD66a) (e.g., CM-24 (MK-6018)); CEA (carcinoembryonic antigen) (e.g., cergutuzumab amunaleukin (RG7813, RO-6895882), RG7802 (R06958688)); CD47 (e.g., HuF9-G4, CC-90002, TTI-621, ALX148, NI-1701, NI-1801, SRF231, and Effi-DEM); PVRIG (poliovirus receptor related immunoglobulin domain containing, CD122R) (e.g., COM701); GARP (glycoprotein A repetitions predominant) (e.g., ARGX-115); CD80 (e.g., galiximab (IDEC-114) and AV 1142742 (RhuDex); CD86; and CD96.

In some aspects, the immune checkpoint inhibitor is an agonist of STING (stimulator of IFN genes) (e.g., 2′ or 3′-mono-fluoro substituted, or 2′3′-di-fluoro substituted mixed linkage 2′,5′-3′,5′ cyclic-di-nucleotides (PCT Publication No. WO 2017/075477 A1); 2′-fluoro substituted, bis-3′,5′ cyclic-di-nucleotides and 2′,2″-diF-Rp,Rp,bis-3′,5′ cyclic-di-nucleotides (PCT Publication No. WO 2016/145102 A1); and Fluorinated cyclic-di-nucleotides (PCT Publication No. WO 2016/096174 A1); or CD20 (e.g., RITUXAN© and ABP 798).

As the skilled person will know, alternative and/or equivalent names may be in use for certain antibodies mentioned above. Such alternative and/or equivalent names are interchangeable in the context of the present invention.

### VI. Administration

An anti-Claudin-1 antibody, a biologically active fragment thereof, or an anti-Claudin-1 CAR T-cell (optionally after formulation with one or more appropriate pharmaceutically acceptable carriers or excipients), in a desired dosage, can be administered to a subject in need thereof by any suitable route. In some aspects, the anti-Claudin-1 antibody, the biologically active fragment thereof, or the anti-Claudin-1 CAR T-cell, is delivered in combination with an immune checkpoint inhibitor, as described above. Various delivery systems are known and can be used to administer antibodies, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, and oral routes. An anti-Claudin-1 antibody, a biologically active fragment thereof, an anti-Claudin-1 CAR T-cell or a pharmaceutical composition thereof, and the immune checkpoint inhibitor may be administered by any convenient or other appropriate route, for example, by infusion or bolus injection, by absorption through epithelial or mucosa linings (e.g., oral mucosa, bronchial mucosa, rectal and intestinal mucosa, etc.). Administration can be systemic or local. As will be appreciated by those of ordinary skill in the art, in aspects where an antibody is administered in combination with an additional therapeutic agent (e.g., an immune checkpoint inhibitor), the antibody and therapeutic agent may be administered by the same route (e.g., intravenously) or by different routes (e.g., intravenously, orally, or subcutaneously).

In some aspects, the anti-Claudin-1 antibody, or the anti-Claudin-1 T-cell, and the immune checkpoint inhibitor are administered intratumorally, intravenously, intraperitoneally, intramuscularly, intrathecally or subcutaneously.

In some aspects, the anti-Claudin-1 antibody, or the anti-Claudin-1 CAR T-cell, is administered prior to the administration of the immune checkpoint inhibitor.

In some aspects, the anti-Claudin-1 antibody, or the anti-Claudin-1 CAR T-cell, and the immune checkpoint inhibitor are administered simultaneously or sequentially.

In some aspects, the anti-Claudin-1 antibody, or the anti-Claudin-1 CAR T-cell, and the immune checkpoint inhibitor are administered in the same composition.

In some aspects, the anti-Claudin-1 antibody, or the anti-Claudin-1 CAR T-cell, and the immune checkpoint inhibitor are administered in different compositions.

An anti-Claudin-1 antibody, or a biologically active fragment thereof, (optionally after formulation with one or more appropriate pharmaceutically acceptable carriers or excipients), or an anti-Claudin-1 CAR T-cell, will be administered in a dosage such that the amount delivered is effective for the intended purpose. The route of administration, formulation and dosage administered will depend on the therapeutic effect desired, the severity of the condition to be treated if already present, the presence of any infection, the age, sex, weight, and general health condition of the patient as well as upon the potency, bioavailability, and in vivo half-life of the antibody or composition used, the use (or not) of concomitant therapies, and other clinical factors. These factors are readily determinable by the attending physician in the course of the therapy. Alternatively or additionally, the dosage to be administered can be determined from studies using animal models (e.g., non-human primates or rodents). Adjusting the dose to achieve maximal efficacy based on these or other methods are well known in the art and are within the capabilities of trained physicians. As studies are conducted using anti-Claudin-1 antibodies, or an anti-Claudin-1 CAR T-cell, further information will emerge regarding the appropriate dosage levels and duration of treatment.

A treatment according to the present invention may consist of a single dose or multiple doses. Thus, administration of an anti-Claudin-1 antibody, or a biologically active fragment thereof, or an anti-Claudin-1 CAR T-cell (or a pharmaceutical composition thereof), may be constant for a certain period of time or periodic and at specific intervals, e.g., hourly, daily, weekly (or at some other multiple day interval), monthly, yearly (e.g., in a time release form). Alternatively, the delivery may occur at multiple times during a given time period, e.g., two or more times per week; two or more times per month, and the like. The delivery may be continuous delivery for a period of time, e.g., intravenous delivery.

In general, the amount of anti-Claudin-1 antibody, or a biologically active fragment thereof, or the anti-Claudin-1 CAR T-cell, (or a pharmaceutical composition thereof) administered will preferably be in the range of about 1 ng/kg to about 100 mg/kg body weight of the subject, for example, between about 100 ng/kg and about 50 mg/kg body weight of the subject; or between about 1 μg/kg and about 10 mg/kg body weight of the subject, or between about 100 μg/kg and about 1 mg/kg body weight of the subject.

### VII. Pharmaceutical Compositions

As mentioned above, anti-Claudin-1 antibodies (and related molecules), or the anti-Claudin-1 CAR T-cell, may be administered per se or as a pharmaceutical composition. Accordingly, the present invention provides pharmaceutical compositions comprising an effective amount of an anti-Claudin-1 antibody, or a biologically active fragment thereof, described herein and at least one pharmaceutically acceptable carrier or excipient. In some aspects, the composition further comprises one or more additional biologically active agents. In some aspects, the one or more additional biologically active agents is an immune checkpoint inhibitor.

In some aspects, the pharmaceutical composition is for use in a method of promoting T cell mediated anti-tumor activity in a subject having a fibrotic tumor.

In some aspects, the pharmaceutical composition is for use in a method of treating cancer in a subject having a solid tumor.

In some aspects, the pharmaceutical composition is for use in a method of increasing therapeutic efficacy of an immune checkpoint inhibitor in a subject having a fibrotic tumor.

The pharmaceutical compositions may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. In some aspects, the anti-Claudin-1 antibody and immune checkpoint inhibitor are formulated together. In some aspects, the anti-Claudin-1 antibody and immune checkpoint inhibitor are formulated separately. It will be understood, however, that the total daily dosage of the compositions will be decided by the attending physician within the scope of sound medical judgement.

### VIII. Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of an anti-Claudin-1 antibody, or a biologically active fragment thereof, or the anti-Claudin-1 CAR T-cell.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kits will preferably be maintained in close confinement for commercial sale.

In some aspects, a pharmaceutical pack or kit includes one or more additional therapeutic agent(s) as described above. Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

### EXAMPLES

The following examples are illustrative and do not limit the scope of the claimed aspects.

### Example 1. Claudin-1 Expression in the Tumor Microenvironment

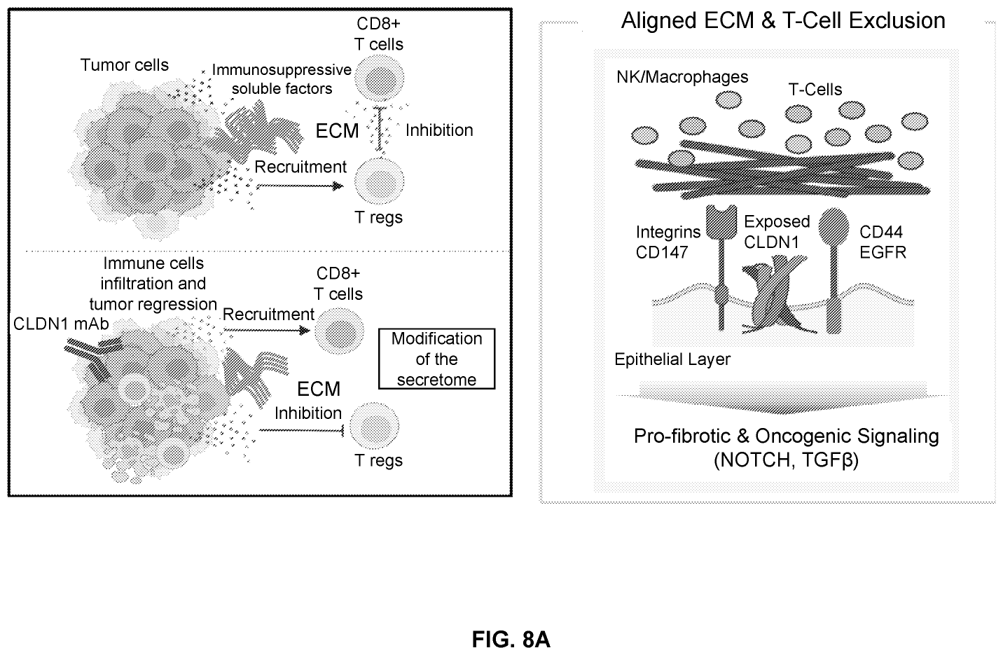

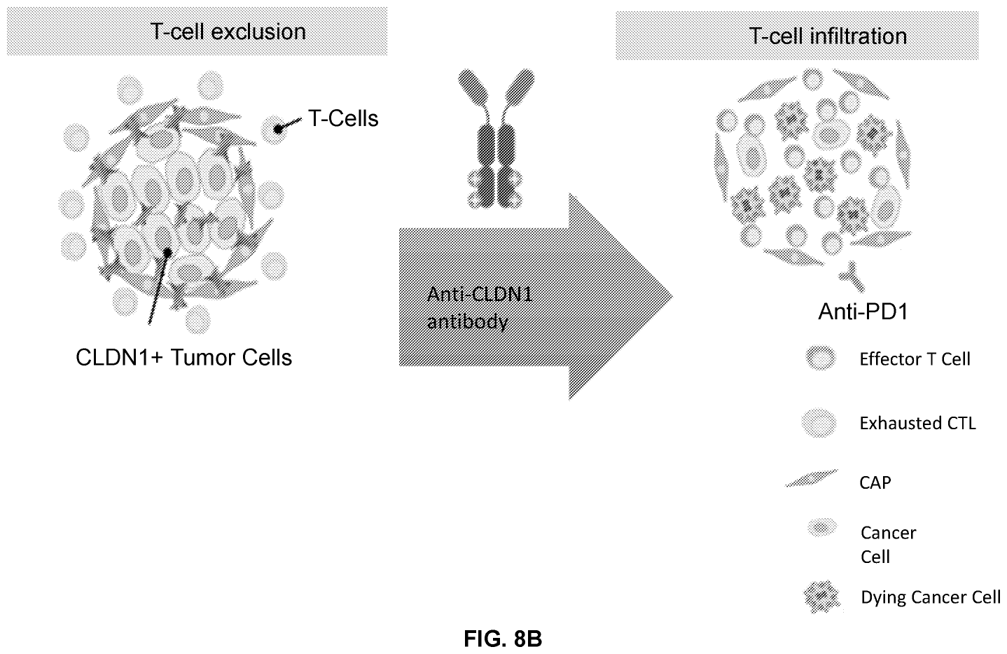

High CLDN1 expression is associated with an immune-low or inactive tumor microenvironment. The change of the tumor cell phenotype by mAb treatment leads to activation of T-cells in the tumor microenvironment likely by alterations in the tumor cell secretome and/or metalloproteinases (FIG. 8A). FIG. 8B shows the mechanism by which administration of an anti-CLDN1 antibody is able to break up the tumor barrier to help transition the tumor state from excluding T cells to allowing T cell infiltration.

### Example 2. Claudin-1 Expression in Fibrotic Tumor Types

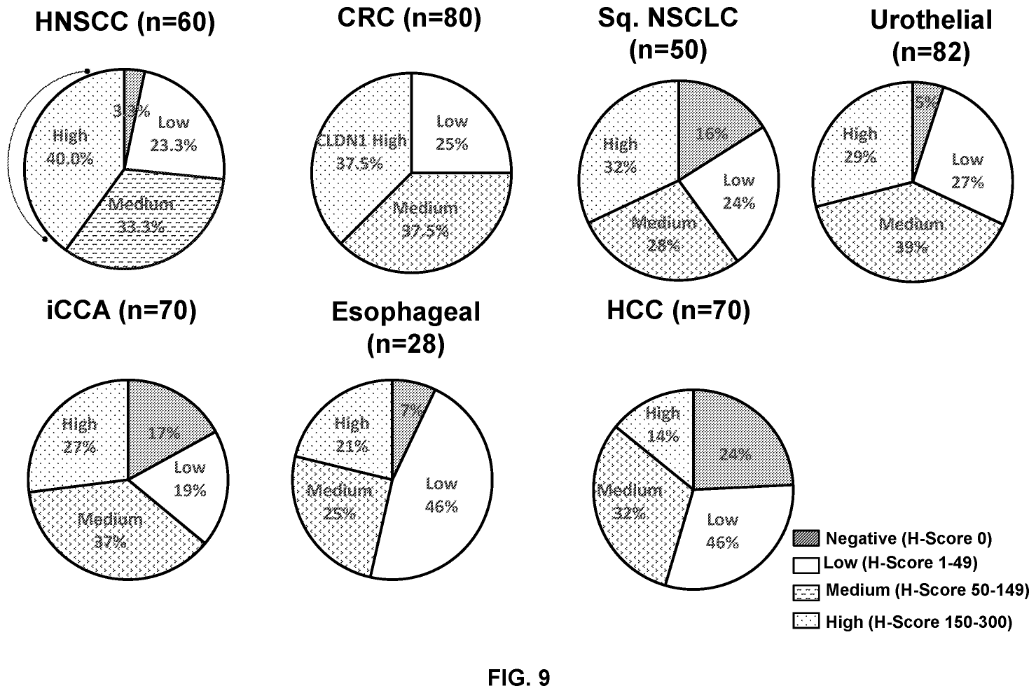

More than 1200 paraffin-embedded tumor biopsies from 12 different indications were stained via immunohistochemistry, analyzing the expression of Claudin-1 (CLDN1), T cell markers (CD3), and fibrosis (Sirius Red staining) in Head and Neck Squamous Cell Carcinoma (HNSCC), Colorectal Cancer (CRC), Esophageal Cancer, Squamous Non-Small Cell Lung Cancer (Sq. NSCLC), Intrahepatic Cholangiocarcinoma (iCCA), Hepatocellular Carcinoma (HCC), and Urothelial Cancer (See FIG. 9). Samples were scored using the semi-quantitative H-score method, which calculates the sum of the percentage and intensity of positively stained tumor cells within the invasive tissue component (negative staining=0; weak staining=1+; moderate staining=2+; strong staining=3+), where a score of 0 represents a negative staining, a score of 1-49 represents low expression, a score of 50-149 represents medium expression, and a score of 150-300 represents a high expression (see e.g., Parris, Toshima Z et al., BMC cancer vol. 14:324 (2014)).

Slides were stained with a Roche Discovery Ultra autostainer. Slides were baked at 60° C. for 1 hour followed by deparaffinization using standard autostainer protocol. Heat-Induced Epitope Retrieval (HIER) was performed using Roche CC1 (high pH) for 48 minutes at 95° C. Peroxidase inhibitor was applied. The primary antibody (anti-CLDN1, Sigma-Aldrich HPA048319 at 1:50) was incubated for 24 minutes at 37° C. The secondary antibody Roche anti-Rabbit HQ was incubated for 8 minutes at 37° C. followed by Roche anti-HQ HRP for 8 minutes at 37° C. DAB was applied using the Roche ChromoMap DAB kit, followed by Roche Hematoxylin II.

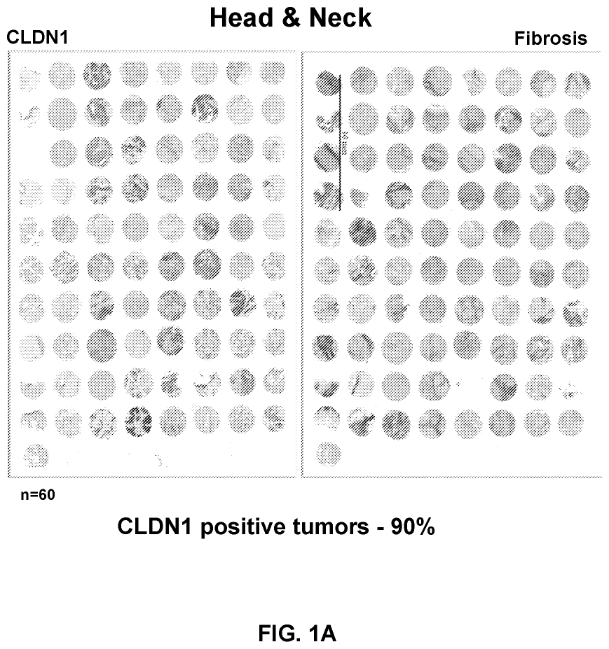

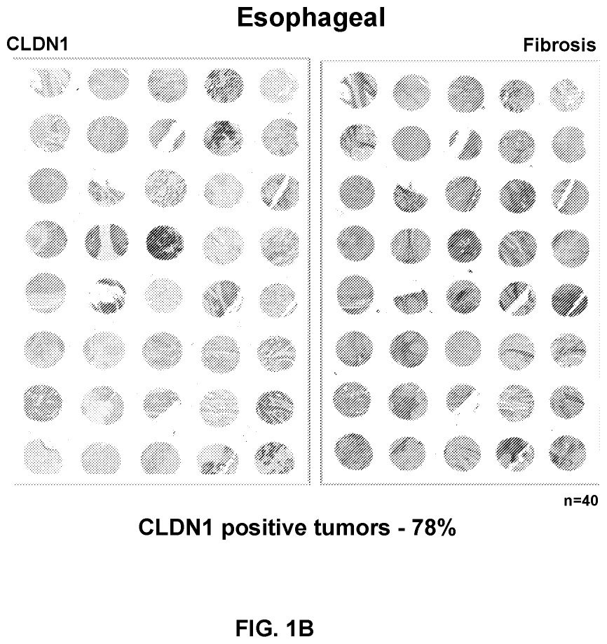

In Head & Neck tumors, 60 samples were analyzed and 90% were determined to be CLDN1 positive tumors (FIG. 1A). In Esophageal Cancer tumors, 40 samples were analyzed and 78% were determined to be CLDN1 positive tumors (FIG. 1). Ultimately, it was determined that CLDN1 is overexpressed in different fibrotic tumors beyond liver cancer.

Furthermore, the data shows that non-junctional CLDN1 (NJ-CLDN1) is frequently overexpressed in solid tumors. Notably, CLDN1 expression on tumor cells positively correlates with the localization of T-cells into a fibrotic tissue environment. T-cell exclusion is one of the mechanisms described to hinder the efficacy of checkpoint inhibitors (CPIs).

### Example 3. Claudin-1 Expression and T Cell Exclusion in Head and Neck Cancer

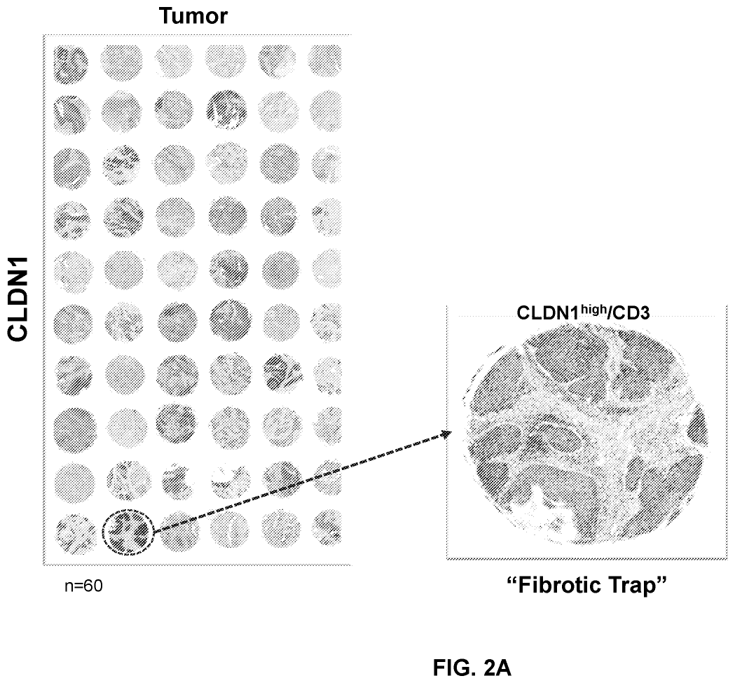

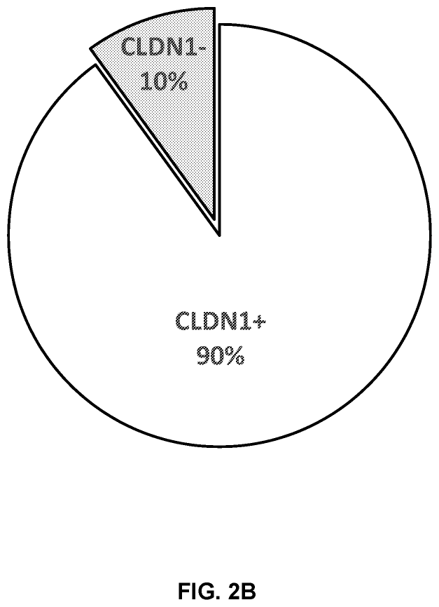

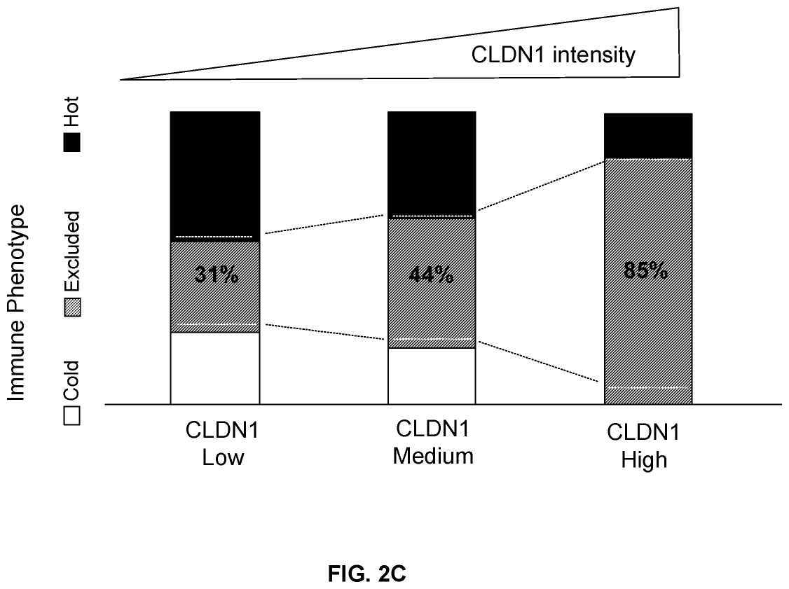

Tumor tissue samples from subjects with Head and Neck cancer were obtained. Immunohistochemistry was used to measure expression of CLDN1 and CD3 (representing fibrosis and a “fibrotic trap”) (FIG. 2A). 90% of tumor samples analyzed were CLDN1 positive (FIG. 2B). FIG. 2C shows the breakdown of immune phenotypes for tumors with varying levels of CLDN1 expression. The immune phenotypes are hot (immune cells in the stroma and between cancer cells), excluded (immune cells within the tumor, but only in the stroma) and cold (almost no immune cells visible). This data shows that T cell exclusion is the main immune phenotype in Head and Neck cancer, with 30-80% of CLDN1 positive tumors having a T cell excluded phenotype.

### Example 4. Claudin-1 Expression and T Cell Exclusion in Esophageal Cancer

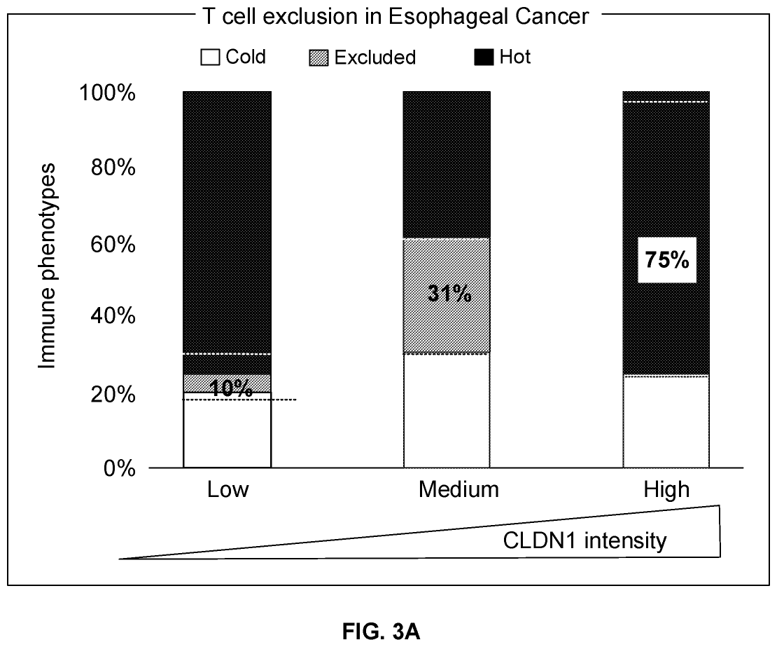

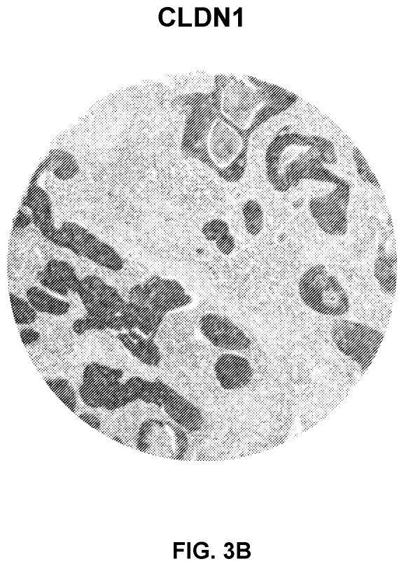

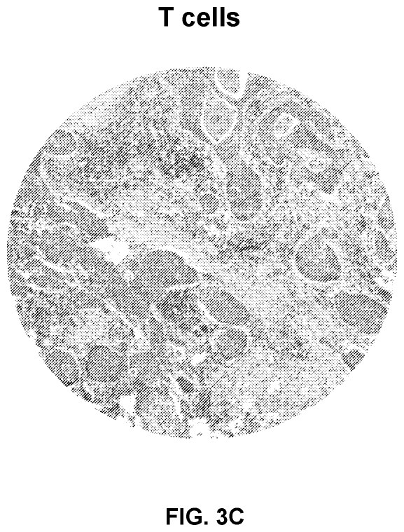

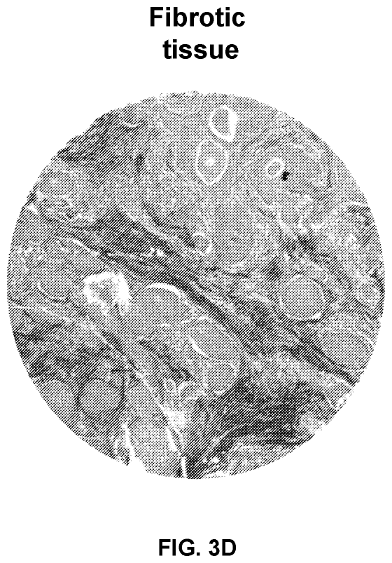

Tumor tissue samples from subjects with Esophageal cancer were obtained. FIG. 3A shows the immune phenotypes for tumors with various levels of CLDN1 expression. The immune phenotypes are hot (immune cells in the stroma and between cancer cells), excluded (immune cells within the tumor, but only in the stroma) and cold (almost no immune cells visible). Immunohistochemistry was used to measure CLDN1 expression (FIG. 3B), T cell presence (FIG. 3C), and fibrotic tissue (FIG. 3D). Taken together, the data shows that CLDN1 expression correlates with T cell exclusion in Cancer.

### Example 5. Overexpression of Mouse Claudin-1 Harboring a Human Extracellular Loop Drives Immune Escape and T Cell Exclusion in Liver Mouse Tumor Cells Hepa1-6 In Vivo

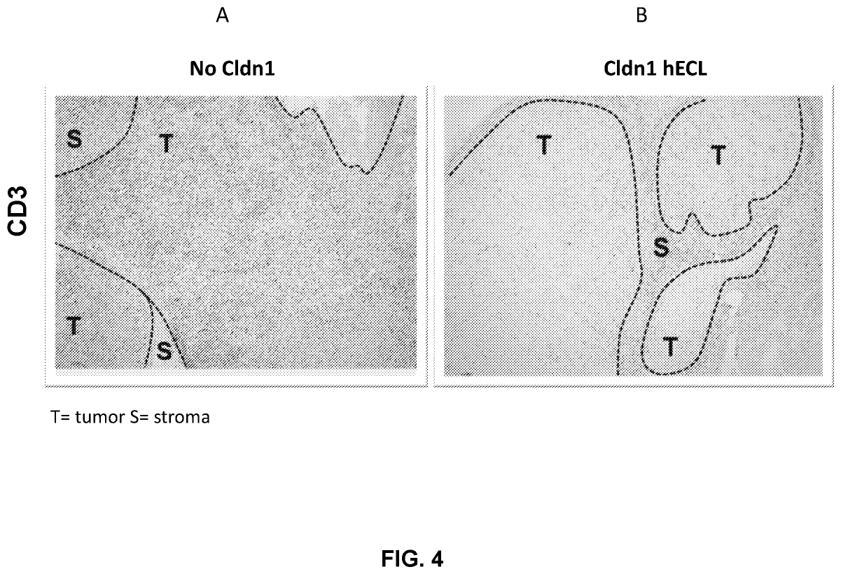

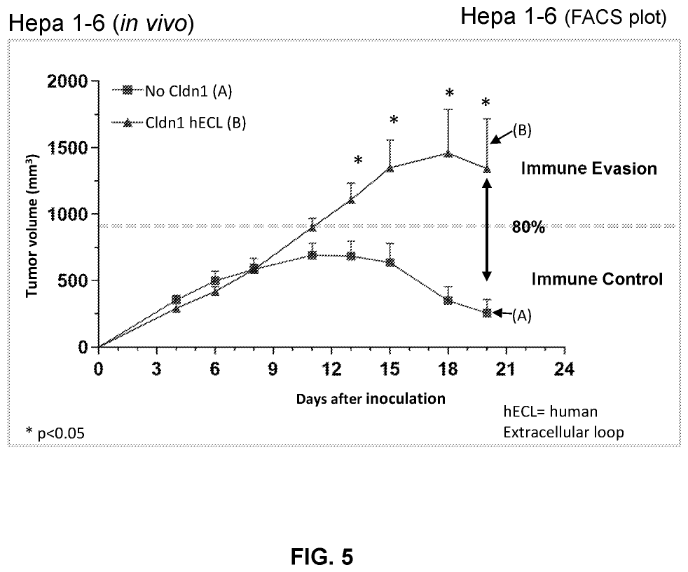

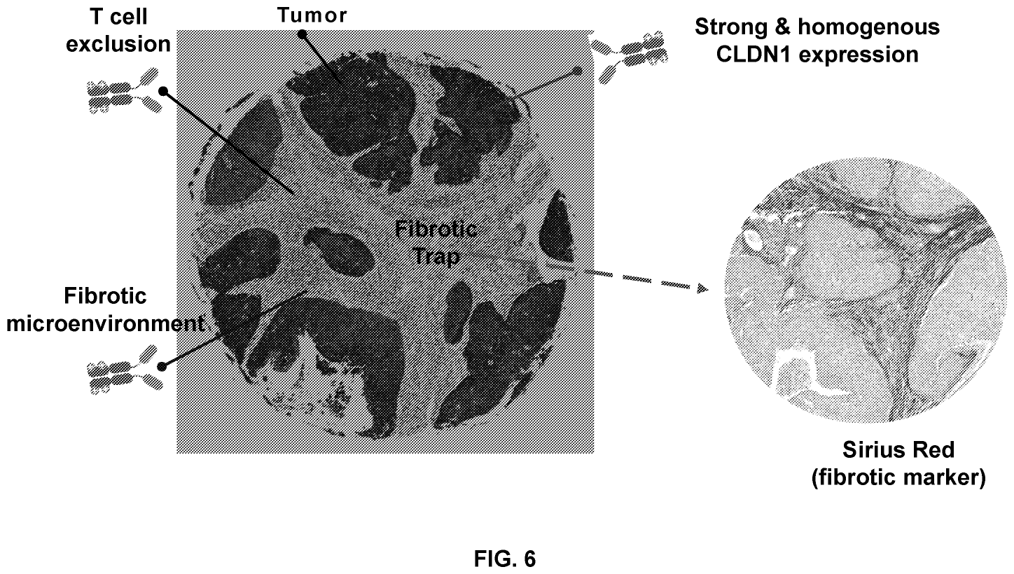

The data provided in FIG. 4A, FIG. 4B, and FIG. 5 show the direct role of the overexpression of Claudin-1 in driving immune evasion and T cell exclusion in vivo. In wild type Hepa 1-6 tumor cells (No Claudin-1; FIG. 5, line with square markers), tumors are rejected by the immune system over time while the overexpression of Claudin-1 (Claudin-1 hECL; FIG. 5, line with triangle markers) drives immune evasion and tumor growth. Anti-CD3 (T cell marker) IHC analysis performed on the tumor sample taken at the end of the experiment (Day 20) shows how overexpression of Claudin-1 (Cldn1 hECL) drives T cell exclusion from the tumor bed and accumulation in the stroma (FIG. 4B) compared to no expression of Claudin-1 (FIG. 4A).

### Example 6. Using Anti-Claudin-1 Antibodies to Break Checkpoint Inhibitor Resistance in Cancer

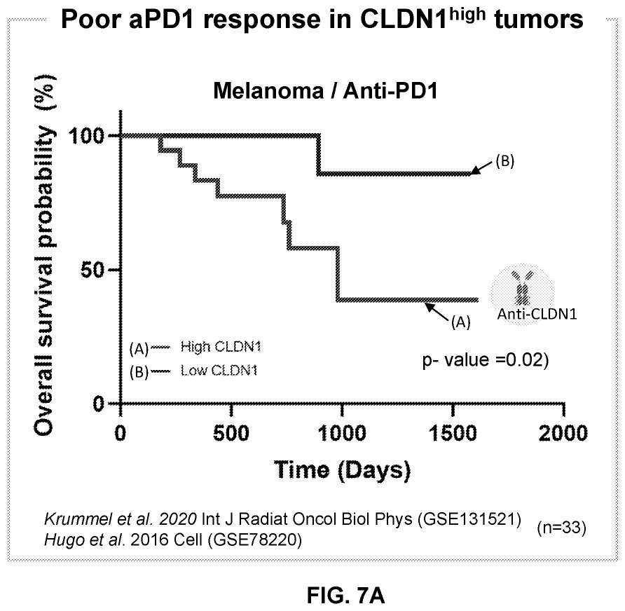

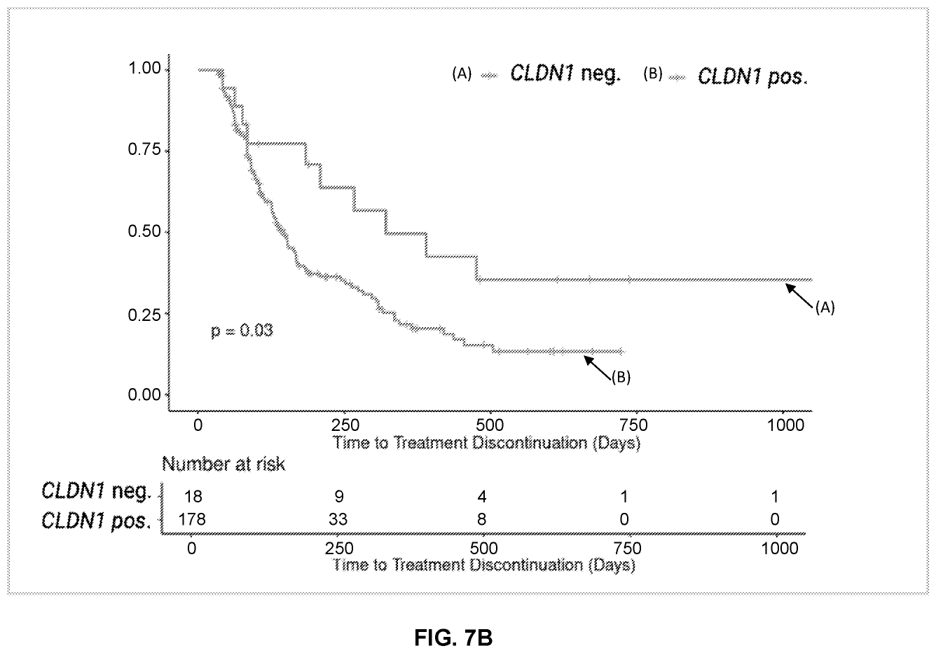

The data provided in Examples 1-4 show that fibrosis is a common denominator for checkpoint inhibitor resistance and T cell exclusion in cancer. Anti-Claudin-1 antibodies, such as any of those described herein, are administered to subjects with a fibrotic tumor. Anti-Claudin-1 antibodies have a direct anti-fibrotic effect, which promotes T cell mediated anti-tumor activity. Importantly, tumors, such as Melanoma (FIG. 7A) and Head and Neck Squamous Cell Carcinoma (FIG. 7B) with high levels of CLDN1 poorly respond to the checkpoint inhibitor aPD1. Anti-Claudin-1 antibodies will be administered in combination with checkpoint inhibitor aPD1 in a Hepa1-6 synergistic tumor model of liver cancer, in which overexpression of mouse CLDN1 drives immune escape in vivo. Additionally, a patient-derived xenograft (PDX) model for Head & Neck cancer, in which high expression of CLDN1 and T cell exclusion have been confirmed, will be used to show the synergistic effect of administering Anti-Claudin-1 antibodies with the checkpoint inhibitor aPD1. Administration of an immune checkpoint inhibitor at the same time or after the administration of the anti-Claudin-1 antibodies allows for increased therapeutic efficacy of immune checkpoint inhibitors in fibrotic tumors.

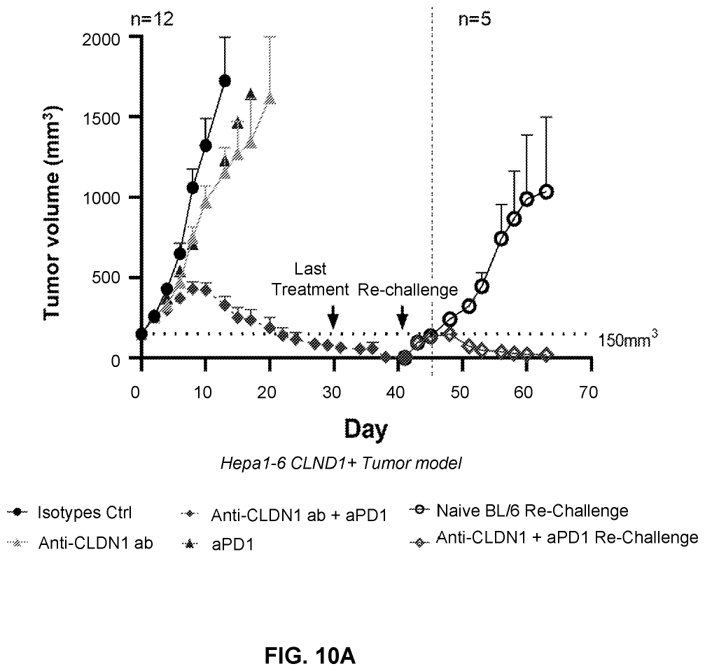

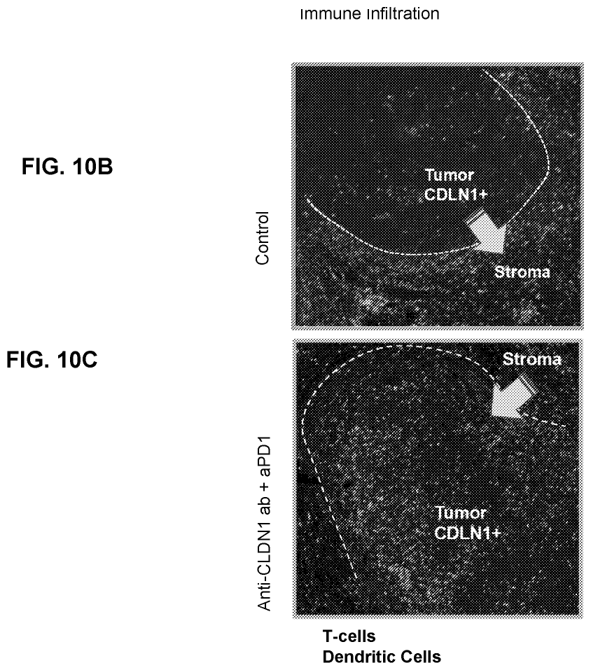

The data in FIG. 10A-10C shows that the overexpression of CLDN1 in Hepa1-6 mouse liver tumor cells promoted T-cell exclusion and resistance to anti-PD1 treatment. Importantly, the anti-CLDN1 antibody of the present disclosure restored both T-cell infiltration and anti-PD1 efficacy in Hepa1-6 CLDN1+ tumors. FIG. 10A shows that tumor volume drastically decreased in the anti-CLDN1 antibody and PD1 antagonist cohort compared to isotype control, anti-CLDN1 antibody alone, and the PD1 antagonist alone cohorts. FIG. 10B and FIG. 10C show that T cells were able to successfully infiltrate CLDN1+ tumors in far greater numbers after administration of the anti-CLDN1 antibody and PD1 antagonist combination.

Mechanistically, NJ-CLDN1 interacts with different components involved in extracellular matrix remodeling, thus establishing a physical barrier that excludes immune cells from the tumor nest. The anti-CLDN1 antibody has a direct antifibrotic effect that perturbs the interface between CDLN1+ tumor cells and the stroma, thus restoring immune cell infiltration.

### Example 7. Generation of Anti-Claudin-1 CAR T Cells (Prophetic)

### Isolation and Activation of T Cells

Patients will be connected to a device that moves peripheral blood through a single-use disposable tubing set. Centrifugal force, guided by optical sensors, will separate the blood into appropriate density bands for isolation and collection of the desired cell layer. Uncollected blood components will be then returned to the patient. Alternatively, apheresis will be used as a method for T cell collection. Engineered T-cells will be isolated from a patients and re-introduced into the same individual (autologous therapy), or isolated from a donor and then introduced into a different individual (heterologous therapy). Collected cells will be cryo-conserved or processed without previous freezing. Isolated cells will be optionally processed for T-cells (specific T-cell subsets) enriching using antibody-conjugated magnetic beads for positive or negative selection. For example, T cells may be enriched based on the expression of CD62L, CD4, and CD8. Isolated T cells will be activated for example via polyclonal stimulation using soluble anti-CD3 antibodies or immobilized CD3 and CD28 antibodies. CD3 and CD28 antibodies may be immobilized by coating tissue culture flasks. Paramagnetic beads, such as Dynabeads, can also be coated with these antibodies; in suspension, the coated beads provide appropriate stimulation for much larger T-cell cultures. Prior to formulating the final cellular products, the beads will be removed, as they could pose a hazard if infused into the patient. Removal will be achieved by disrupting the T-cell/bead aggregates via agitation and then passing the suspension through a strong magnetic field, which retains the beads but allows cells to flow through. Alternatively, stimulation reagents such as Transact will be employed, which utilizes humanized anti-CD3 and anti-CD28 antibodies conjugated to a colloidal polymeric nanomatrix. The nanomatrix will be washed out in a centrifugation step, prior to final product formulation. Alternatively, a similar method of T-cell stimulation using a hydrogel “stimulation matrix” incorporating antibodies, which can also be removed by washing after stimulation and expansion, will be used. Other approaches, such as soluble activation proteins, lipid microbubbles, dissolvable microspheres, and linked antibodies are also potential options which will be used to activate the isolated T-cells.

### Genetic Engineering of Isolated T Cells to Achieve Expression of the Anti-Claudin-1 CAR

After activation, T cells will be genetically engineered to express the anti-Claudin-1 CAR. Isolated T cells will be genetically engineered suing viral systems, or alternatively non-viral systems. Plasmid-based transposon/transposase systems and viral vectors, including, but not limited to, gamma-retroviral and lentiviral vectors as well as genome editing (e.g., CRISPR/Cas9-based gene editing) and electroporation of naked DNA will be applied for gene delivery on the anti-Claudin-1 coding region, and associated regulatory sequences, into the isolated T cells.

### Expansion of Anti-Claudin-1 CAR T-Cells

Engineered CAR T-cells expressing the anti-Claudin-1 CAR will then be expanded in vitro, by standard culturing techniques, or by alternative methods comprising rocking motion bioreactors, such as the Xuri™ Cell Expansion System and WAVE™ Bioreactor System, which utilize a perfusion regime to add nutrients as well as remove growth-inhibiting substances, thereby simplifying the manufacturing process. Engineered anti-Claudin-1 CAR T-cells will also be stimulated with supplemented γ-chain cytokines including, but not limited to, IL-2, IL-4, IL-7, IL-9, IL-15, and IL-21. Optionally the anti-Claudin-1 CAR T-cells will also be treated with specific pathway inhibitors, including, but not limited to, GSK3β, mTOR, AKT, and PI3K.

Engineered CAR T-cells expressing the anti-Claudin-1 CAR will then be cryopreserved for quality control tests before administration to the patient.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

What is claimed is:

1. A method of promoting T cell mediated anti-tumor activity in a subject having a fibrotic tumor, comprising administering an anti-Claudin-1 antibody to the subject.

2. The method of claim 1, further comprising administering an immune checkpoint inhibitor.

3. A method of treating cancer in a subject having a solid tumor comprising administering to the subject a therapeutically effective amount of an anti-Claudin-1 antibody and an immune checkpoint inhibitor, wherein the anti-Claudin-1 antibody promotes T cell mediated anti-tumor activity in a tumor in the subject.

4. A method of increasing therapeutic efficacy of an immune checkpoint inhibitor in a subject having a fibrotic tumor, comprising:
a. administering an anti-Claudin-1 antibody to the subject, wherein the anti-Claudin-1 antibody promotes T cell mediated anti-tumor activity in the fibrotic tumor; and
b. administering the immune checkpoint inhibitor to the subject.

5. The method of any one of claims 2-4, wherein the anti-Claudin-1 antibody is administered prior to the administration of the immune checkpoint inhibitor.

6. The method of any one of claims 2-4, wherein the anti-Claudin-1 antibody and the immune checkpoint inhibitor are administered simultaneously or sequentially.

7. The method of any one of claims 2-4, wherein the anti-Claudin-1 antibody and the immune checkpoint inhibitor are administered in the same composition.

8. The method of any one of claims 2-4, wherein the anti-Claudin-1 antibody and the immune checkpoint inhibitor are administered in different compositions.

9. The method of any one of claims 2-4, wherein the anti-Claudin-1 antibody and/or the immune checkpoint inhibitor are administered intratumorally, intravenously, intraperitoneally, intramuscularly, intrathecally or subcutaneously.

10. The method of any one of claims 2-4, wherein the immune checkpoint inhibitor is an antagonist of PD-1, PD-L1, CTLA-4, LAG-3, TIM-3, TIGIT, VISTA, B7-H3, BTLA, and/or Siglec-15.

11. The method of any one of claims 2-4, wherein the immune checkpoint inhibitor is a small molecule inhibitor.

12. The method of any one of claims 2-4, wherein the immune checkpoint inhibitor is an antibody.

13. The method of claim 12, wherein the immune checkpoint inhibitor is a PD-1 antagonist selected from the group consisting of nivolumab, pembrolizumab, cemiplimab, and dostarlimab.

14. The method of claim 12, wherein the immune checkpoint inhibitor is a PD-L1 antagonist selected from the group consisting of atezolizumab, durvalumab, and avelumab.

15. The method of claim 12, wherein the immune checkpoint inhibitor is a CTLA-4 antagonist is selected from the group consisting of ipilimumab and tremelimumab.

16. The method of claim 12, wherein the immune checkpoint inhibitor is a TIGIT antagonist selected from the group consisting of tiragolumab, ociperlimab, domvanalimab, etigilimab, and vibostolimab.

17. The method of claim 3, wherein the cancer comprises a fibrotic tumor.

18. The method of any one of claims 1, 2, or 4-17, wherein the fibrotic tumor is characterized by a high expression of Claudin-1 relative to a reference sample.

19. The method of claim 18, wherein the reference sample is a tissue sample from a normal tissue, wherein the normal tissue is adjacent to the tumor.

20. The method of any one of claims 1-19, wherein the tumor is selected from the group consisting of a head and neck, a lung, a breast, a melanoma, a colorectal, a pancreatic, an esophageal, a cholangiocarcinoma, and a hepatocellular tumor.

21. The method of any one claims 1-20, wherein the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

22. The method of any one of claims 1-21, wherein the anti-Claudin-1 antibody is humanized.

23. The method of any one of claims 1-22, wherein the anti-Claudin-1 antibody comprises a VH comprising the amino acid sequence set forth in SEQ ID NO: 3 or SEQ ID NO: 13.

24. The method of any one of claims 1-23, wherein the anti-Claudin-1 antibody comprises a VL comprising the amino acid sequence set forth in SEQ ID NO: 4 or SEQ ID NO: 14.

25. The method of any one of claims 1-24, wherein the anti-Claudin-1 antibody comprises a VH comprising the amino acid sequence set forth in SEQ ID NO: 3; and a VL comprising the amino acid sequence set forth in SEQ ID NO: 4.

26. The method of any one of claims 1-25, wherein the anti-Claudin-1 antibody comprises a VH comprising the amino acid sequence set forth in SEQ ID NO: 13; and a VL comprising the amino acid sequence set forth in SEQ ID NO: 14.

27. The method of any one of claims 1-26, wherein the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

28. The method of any one of claims 1-27, wherein the anti-Claudin-1 antibody comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

29. The method of any one of claims 1-28, wherein the anti-Claudin-1 antibody comprises a heavy chain sequence comprising the amino acid sequence set forth as SEQ ID NO: 1.

30. The method of any one of claims 1-29, wherein the anti-Claudin-1 antibody comprises a light chain sequence comprising the amino acid sequence set forth as SEQ ID NO: 2.

31. A method of promoting T cell mediated anti-tumor activity in a subject having a fibrotic tumor, comprising administering an anti-Claudin-1 chimeric antigen receptor (CAR) T cell to the subject.

32. The method of claim 31, further comprising administering an immune checkpoint inhibitor.

33. A method of treating cancer in a subject having a solid tumor comprising administering to the subject a therapeutically effective amount of an anti-Claudin-1 CAR T Cell and an immune checkpoint inhibitor, wherein the anti-Claudin-1 CAR T Cell promotes T cell mediated anti-tumor activity in a tumor in the subject.

34. A method of increasing therapeutic efficacy of an immune checkpoint inhibitor in a subject having a fibrotic tumor, comprising:
a. administering an anti-Claudin-1 CAR T Cell to the subject, wherein the anti-Claudin-1 CAR T Cell promotes T cell mediated anti-tumor activity in the fibrotic tumor; and
b. administering the immune checkpoint inhibitor to the subject.
